(19) United States
(12) Patent Application Publication
(10) Pub. No.: US 2023/0258629 A1
(43) Pub. Date: Aug. 17, 2023

(54) EXTRACELLULAR VESICLE PURIFICATION MATERIAL AND PURIFICATION METHOD

(71) Applicant: Beijing Tongbio Technologies Inc., Beijing City (CN)

(72) Inventor: Zhihui Xu, Hohhot (CN)

(21) Appl. No.: 17/740,283

(22) Filed: May 9, 2022

(30) Foreign Application Priority Data
Feb. 11, 2022 (CN) 202210126779.6

(51) Int. Cl. G01N 33/543; G01N 1/40; C12N 15/10

(52) U.S. Cl. CPC G01N 33/5434; G01N 1/405; C12N 15/1013; G01N 2405/06; G01N 2446/20

(57) ABSTRACT

The present disclosure relates to an extracellular vesicle purification material and purification method. The purification method adopts metal oxide microspheres or magnetic beads that can reversibly bind to phosphatidylserine to purify extracellular vesicles, and the purification material comprises nano zirconium dioxide microspheres, nano titanium dioxide microspheres or nano aluminum oxide microspheres, nano zirconium dioxide magnetic microspheres, nano titanium dioxide magnetic microspheres or nano aluminum oxide magnetic microspheres, and porous zirconium dioxide nano microspheres, porous titanium dioxide nano microspheres or porous aluminum oxide nano microspheres. The purification method of the present disclosure can quickly purify multiple samples: serum/plasma, urine and cell culture supernatant, has a moderate flux so as to retain extracellular vesicles to the greatest extent, and can obtain high-purity extracellular vesicles, without chelating agents and other reagents that may affect subsequent experiments, via elution.

### TECHNICAL FIELD

The present disclosure belongs to the technical field of biological technologies, particularly to an extracellular vesicle purification material and purification method.

### BACKGROUND

Extracellular vesicles (EVs) refer to vesicular bodies with a double-layer membrane structure, which fall off from cytomembranes or are secreted by cells and have a diameter ranging from 10 nm to 1000 nm. The extracellular vesicles consist of microvesicles (MVs) and exosomes (Exs), and MVs are small vesicles falling off from cytomembranes after activation, damage or apoptosis of cells and have a diameter of about 100 nm-1000 nm. Exs are released to the exterior of the cells in an exocrine form after multivesicular bodies are fused with cytomembranes and have a diameter of about 10 nm-200 nm. EVs are secreted from most of cell types, and exist in body fluids (including plasma, serum, urine and cerebrospinal fluid). EVs can act as a natural vector to be used for delivering macromolecules between cells, including proteins, nucleic acids (such as DNA and RNA), biological active substances and carbohydrates. It has been shown that the signaling of EVs plays an important role in various physiological and pathological conditions (including carcinogenic diseases, neurodegenerative diseases, cardiovascular diseases, autoimmune diseases and metabolic diseases). The surfaces of EVs contain markers (proteins/carbohydrates) inherited from the surfaces of origin cells, which allow for classification and targeting of cells and tissue-specific EVs. Inclusions (including but not limited to proteins and RNA) of EVs depend on their origin cells, donor pathophysiological conditions, cells conditions such as oxidative or metabolic stress and donor's response to therapeutic intervention, and therefore can act as origins of biological markers of specific body or disease conditions. Blood circulates throughout the body, and is collected at regular intervals in clinical practices so that it becomes an ideal origin of a biological marker of a disease. Serum and plasma as well as fluid and a cell-free phase of blood contain EVs and other particles, circulated free proteins, circulated free DNA and lipids. Abundance of some components (such as proteins and a complement system) in a plasma sample makes detection of rare metabolites and proteins challenging. Protein detection is mainly based on antibody-antigen interaction, which is generally referred to as immunoassay, and used in traditional methods such as gel electrophoresis (western blotting), enzyme-linked immunosorbent assay (ELISA) and immunochemiluminescence, as well as new digital methods such as single molecule array (Simoa) and Erenna. The immunoassay performance depends on the quality of the antibody (sensitivity and specificity), irrespective of detection methods. Antibody-antigen interaction characteristics depend on antibody specificity and interaction environments including buffer concentrations of salts and proteins, pH and antigen availability. Plasma and serum enrich different components, such as proteins and metabolites, and the complicated environment having rich proteins is a bad environment for protein immunoassay, especially if the abundance of a target analyte is relatively low. Use of EVs needs to overcome this problem, because the purification of EVs or a subpopulation of EVs will reduce the complicacy of samples, and allows for detecting low-abundance targets or epitopes from the sample. In addition, RNA in biological fluids (such as plasma or serum) is essentially unstable, and undergoes various types of RNA enzymes digesting it. EVs carry cell source relevant multiple proteins, lipids, DNA, mRNA, miRNA, etc., and participate in processes such as intercellular communication, cell migration, angiogenesis and immunoregulation. The raised level of extracellular vesicles is found in diabetes, cardiovascular disease, AIDS, chronic inflammatory diseases and cancer, thus they are likely to become diagnostic markers for this type of diseases. Therefore, it is extremely important for accurate qualitative and quantitative researches on extracellular vesicles.

At present, there are mainly two strategies for large-scale purification of extracellular vesicles on the market:

Tangential flow ultrafiltration: the extracellular vesicles in the sample are concentrated through membranes with different pore sizes. Due to adoption of physical filtration, the proteins and other soluble substances can be concentrated as long as their sizes are larger than the pore size, so as to cause impure extracellular vesicles. Tangential flow ultrafiltration can also mechanically shear and damage the extracellular vesicles, resulting in the rupture of the extracellular vesicles so as to reduce the recovery rate of the extracellular vesicles.

Exclusion chromatographic column mode: an exclusion chromatographic resin column is similar to a molecular sieve. Molecules or vesicles with large particle sizes can outflow from the exclusion chromatographic resin column, and then molecules and vesicles with small particle sizes can enter a resin ball to be delayed and then outflow. The extracellular vesicles with appropriate sizes are separated through interception of particle sizes, because the extracellular vesicles are not a particle size, but a mixture of different particle sizes, and the exclusion chromatographic column can lose a part of extracellular vesicles, and meanwhile molecules and soluble substances with the same particle sizes can also be collected. Because of no affinity binding, the exclusion chromatographic column does not have the function of concentration, and finally the volume of the purified extracellular vesicles changes little without the purpose of concentration.

Other methods include isolation of extracellular vesicles by ultracentrifugation with or without density gradient. Although this process produces a relatively pure EV population, it is laborious and inefficient, and generates high differences between samples. The extracellular vesicle products purified by using chemical precipitation are not pure, and therefore, immunoassays and other detection methods are not optimal for isolation of low-purity extracellular vesicles. In addition, these methods enrich many kinds of extracellular vesicles found in plasma, representing a highly heterogeneous population of extracellular vesicles secreted from multiple cell types. Large mixtures of extracellular vesicle types from different cell origins can interfere with immunoassays or over represent protein and RNA characteristics from normal tissues and cells, which mask the profile of disease extracellular vesicles. For these reasons, simple and reproducible methods for isolating total extracellular vesicle populations or purifying specific subsets of extracellular vesicles can significantly enhance the detection of biomarkers based on extracellular vesicle related proteins and microRNA.

In view of this, apply for this patent.

### SUMMARY

In order to solve the problems existing in the prior art, the present disclosure provides an extracellular vesicle purification material and purification method, with a fast purification speed and a good purification effect.

The objective of the present disclosure is to provide an extracellular vesicle purification material.

Another objective of the present disclosure is to provide an extracellular vesicle purification method.

According to an extracellular vesicle purification material of embodiments of the present disclosure, the purification material is a metal oxide microsphere or metal oxide magnetic bead that can reversibly bind to phosphatidylserine.

Further, the metal oxide microsphere is a non-magnetic nano microsphere or porous nano microsphere of zirconium dioxide, titanium dioxide or aluminum oxide, and the metal oxide magnetic bead is a magnetic nano microsphere of zirconium dioxide, titanium dioxide or aluminum oxide.

Preferably, the non-magnetic nano microsphere is a nano zirconium dioxide microsphere, a nano titanium dioxide microsphere or a nano aluminum oxide microsphere, the magnetic nano microsphere is a nano zirconium dioxide magnetic microsphere, a nano titanium dioxide magnetic microsphere or a nano aluminum oxide magnetic microsphere, and the nano porous microsphere is a porous zirconium dioxide nano microsphere, a porous titanium dioxide nano microsphere or a porous aluminum oxide nano microsphere.

According to the extracellular vesicle purification material of embodiments of the present disclosure, further, a method for preparing the nano zirconium dioxide microsphere comprises the following steps:

(1) preparation of a zirconium dioxide precursor: mixing and heating zirconium dioxide powders, a first concentrated sulfuric acid and ammonium sulfate until being dissolved, adding a second concentrated sulfuric acid after the obtained mixture is placed at room temperature, and uniformly mixing to form the zirconium dioxide precursor; preferably, a weight-to-volume ratio of the zirconium dioxide powders to the first concentrated sulfuric acid and the second concentrated sulfuric acid being 0.09-0.11 g:9-11 ml:2-4 g:18-22 ml;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml; and

(3) preparation of a nano zirconium dioxide microsphere: introducing nitrogen into the solution A obtained in step (2), adding the zirconium dioxide precursor obtained in step (1) under the state of stirring, then dropwise adding N,N-dimethylformamide, carrying out suction filtration after completion of reaction to obtain a white precipitate, washing the white precipitate with absolute ethyl alcohol, and then drying to obtain the nano zirconium dioxide microsphere; preferably, a volume ratio of the solution A to the zirconium dioxide precursor to N,N-dimethylformamide being 7:3:2.

According to the extracellular vesicle purification material of embodiments of the present disclosure, further, a method for preparing the nano titanium dioxide microsphere comprises the following steps:

(1) preparation of a titanium dioxide precursor: dissolving tetrabutyl titanate into absolute ethyl alcohol, then adding deionized water, sufficiently and uniformly mixing, then adding concentrated hydrochloric acid, sealing, and then placing for 1-3 days at room temperature to obtain a light yellow transparent liquid, namely, the titanium dioxide precursor; preferably, a volume ratio of tetrabutyl titanate to absolute ethyl alcohol to deionized water to concentrated hydrochloric acid being 4:2:50:0.7;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml; and

(3) preparation of a nano titanium dioxide microsphere: introducing nitrogen into the solution A obtained in step (2), adding the titanium dioxide precursor obtained in step (1) under the state of stirring, then dropwise adding N,N-dimethylformamide, carrying out suction filtration after completion of reaction to obtain a white precipitate, washing the white precipitate with absolute ethyl alcohol, and then drying to obtain the nano titanium dioxide microsphere; preferably, a volume ratio of the solution A to the titanium dioxide precursor to N,N-dimethylformamide being 7:3:2.

According to the extracellular vesicle purification material of embodiments of the present disclosure, further, a method for preparing the nano aluminum oxide microsphere comprises the following steps:

(1) preparation of an aluminum oxide precursor: dissolving sodium metaaluminate into deionized water, and sufficiently stirring until being completely dissolved to form a uniform and transparent solution, namely, the aluminum oxide precursor; preferably, a volume ratio of sodium metaaluminate to deionized water being 0.1 g:50 ml;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml; and

(3) preparation of a nano aluminum oxide microsphere: introducing nitrogen into the solution A obtained in step (2), adding the aluminum oxide precursor obtained in step (1) under the state of stirring, then dropwise adding N,N-dimethylformamide, carrying out suction filtration after completion of reaction to obtain a white precipitate, washing the white precipitate with absolute ethyl alcohol, and then drying to obtain the nano aluminum oxide microsphere; preferably, a volume ratio of the solution A to the aluminum oxide precursor to N,N-dimethylformamide being 7:3:2.

In step (3), the time of the reaction is 10-12 h.

According to the extracellular vesicle purification material of embodiments of the present disclosure, further, a method for preparing the nano zirconium dioxide magnetic microsphere comprises the following steps:

(1) preparation of a zirconium dioxide precursor: mixing and heating zirconium dioxide powders, a first concentrated sulfuric acid and ammonium sulfate until being dissolved, adding a second concentrated sulfuric acid after the obtained mixture is placed at room temperature, uniformly mixing to form the zirconium dioxide precursor; preferably, a weight-to-volume ratio of the zirconium dioxide powders to the first concentrated sulfuric acid and the second concentrated sulfuric acid being 0.09-0.11 g:9-11 ml:2-4 g:18-22 ml;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml;

I. preparation of ferroferric oxide microsphere powders: dissolving ferric trichloride and ferrous sulfate into the solution A obtained in step (2), sufficiently stirring until being evenly mixed, dropwise adding a saturated ammonium bicarbonate solution under the state of stirring, meanwhile introducing nitrogen, stirring and heating, dropwise adding sodium hydroxide solution when the temperature is raised to 75-85° C., continuing to stir and mix under the atmosphere of nitrogen after observing that the solution turns pure black, harvesting a black precipitate via magnetic absorption, washing the black precipitate with absolute ethyl alcohol and deionized water in turn, and then drying to form the ferroferric oxide microsphere powders; preferably, the drying temperature being 60° C.; the concentration of the sodium hydroxide solution being 1M; a weight-to-volume ratio of ferric trichloride to ferrous sulfate to saturated ammonium bicarbonate solution to 1M sodium hydroxide solution being 3.24 g:2.78 g:50 ml:10 ml; and

II. preparation of a nano zirconium dioxide magnetic microsphere: adding the ferroferric oxide microsphere powders obtained in step I into the solution A obtained in step (2), introducing nitrogen, adding the zirconium dioxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, magnetically absorbing a brown precipitate, then washing the brown precipitate with absolute ethyl alcohol, and drying to obtain the nano zirconium dioxide magnetic microsphere; preferably, the drying temperature being 100° C.; the drying time being 4 h; a weight-to-volume ratio of the solution A to the ferroferric oxide microsphere powder to the zirconium dioxide precursor to N,N-dimethylformamide being 70 ml:2 g:30 ml:20 ml.

According to the extracellular vesicle purification material of embodiments of the present disclosure, further, a method for preparing the nano titanium dioxide magnetic microsphere comprises the following steps:

(1) preparation of a titanium dioxide precursor: dissolving tetrabutyl titanate into absolute ethyl alcohol, then adding deionized water, sufficiently and uniformly mixing, then adding concentrated hydrochloric acid, sealing, and then placing for 1-3 days at room temperature to obtain a light yellow transparent liquid, namely, titanium dioxide precursor; preferably, a volume ratio of tetrabutyl titanate to absolute ethyl alcohol to deionized water to concentrated hydrochloric acid being 4:2:50:0.7;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml;

I. preparation of ferroferric oxide microsphere powders: dissolving ferric trichloride and ferrous sulfate into the solution A obtained in step (2), sufficiently stirring until being evenly mixed, dropwise adding a saturated ammonium bicarbonate solution under the state of stirring, meanwhile introducing nitrogen, stirring and heating, dropwise adding a sodium hydroxide solution when a temperature is raised to 75-85° C., continuing to stir and mix under the atmosphere of nitrogen after observing that the solution turns pure black, harvesting a black precipitate via magnetic absorption, washing the black precipitate with absolute ethyl alcohol and deionized water in turn, and then drying to form the ferroferric oxide microsphere powders; preferably, the drying temperature being 60° C.; the concentration of the sodium hydroxide solution being 1M; a weight-to-volume ratio of ferric trichloride to ferrous sulfate to saturated ammonium bicarbonate solution to 1M sodium hydroxide solution being 3.24 g:2.78 g:50 ml:10 ml; and

II. preparation of a nano titanium dioxide magnetic microsphere: adding the ferroferric oxide microsphere powders obtained in step I into the solution A obtained in step (2), introducing nitrogen, adding the titanium dioxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, magnetically absorbing a brown precipitate, then washing the brown precipitate with absolute ethyl alcohol, and drying to obtain the nano titanium dioxide magnetic microsphere; preferably, the drying temperature being 100° C.; the drying time being 4 h; a weight-to-volume ratio of the solution A to the ferroferric oxide microsphere powder to the titanium dioxide precursor to N,N-dimethylformamide being 50 ml:2 g:50 ml:20 ml.

According to the extracellular vesicle purification material of embodiments of the present disclosure, further, a method for preparing the nano aluminum oxide magnetic microsphere comprises the following steps:

(1) preparation of an aluminum oxide precursor: dissolving sodium metaaluminate into deionized water, and sufficiently stirring until being completely dissolved to form a uniform and transparent solution, namely, the aluminum oxide precursor; preferably, a volume ratio of sodium metaaluminate to deionized water being 0.1 g:50 ml;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml;

I. preparation of ferroferric oxide microsphere powders: dissolving ferric trichloride and ferrous sulfate into the solution A obtained in step (2), sufficiently stirring until being evenly mixed, dropwise adding a saturated ammonium bicarbonate solution under the state of stirring, meanwhile introducing nitrogen, stirring and heating, dropwise adding a sodium hydroxide solution when a temperature is raised to 75-85° C., continuing to stir and mix under the atmosphere of nitrogen after observing that the solution turns pure black, harvesting a black precipitate via magnetic absorption, washing the black precipitate with absolute ethyl alcohol and deionized water in turn, and then drying to form the ferroferric oxide microsphere powders; preferably, the drying temperature being 60° C.; the concentration of the sodium hydroxide solution being 1M; a weight-to-volume ratio of ferric trichloride to ferrous sulfate to saturated ammonium bicarbonate solution to 1M sodium hydroxide solution being 3.24 g:2.78 g:50 ml:10 ml; and

II. preparation of a nano aluminum oxide magnetic microsphere: adding the ferroferric oxide microsphere powders obtained in step I into the solution A obtained in step (2), introducing nitrogen, adding the aluminum oxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, magnetically absorbing a brown precipitate, then washing the brown precipitate with absolute ethyl alcohol, and drying to obtain the nano aluminum oxide magnetic microsphere; preferably, the drying temperature being 100° C.; the drying time being 4 h; a weight-to-volume ratio of the solution A to the ferroferric oxide microsphere powder to the aluminum oxide precursor to N,N-dimethylformamide being 50 ml:2 g:50 ml:20 ml.

According to the extracellular vesicle purification material of embodiments of the present disclosure, further, a method for preparing the porous zirconium dioxide nano microsphere comprises the following steps:

(1) preparation of a zirconium dioxide precursor: mixing and heating zirconium dioxide powders, a first concentrated sulfuric acid and ammonium sulfate until being dissolved, adding a second concentrated sulfuric acid after the obtained mixture is placed at room temperature, uniformly mixing to form the zirconium dioxide precursor; preferably, a weight-to-volume ratio of the zirconium dioxide powders to the first concentrated sulfuric acid and the second concentrated sulfuric acid being 0.09-0.11 g:9-11 ml:2-4 g:18-22 ml;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml;

i. preparation of polystyrene microsphere powders: adding a first polystyrene and a first ammonium persulfate into the solution A obtained in step (2), sufficiently stirring and emulsifying under the atmosphere of nitrogen, dropwise adding a second polystyrene when stirring and heating to 45-55° C., adding a second ammonium persulfate after completion of dropwise addition, heating to 80-90° C., continuing to stir under the atmosphere of nitrogen until being evenly mixed, cooling to 45-55° C., then dropwise adding a sodium chloride solution, carrying out suction filtration to obtain a white precipitate, then washing the white precipitate with absolute ethyl alcohol and deionized water in turn, and then drying to form the polystyrene microsphere powders; the concentrations of ammonium persulfate being both 200 mg/ml; the concentration of the sodium chloride solution being 2M; the drying temperature being 60° C.; a weight-to-volume ratio of the first polystyrene to the first ammonium persulfate to solution A to the second polystyrene to the second ammonium persulfate to the sodium chloride solution being 30 g:2.5 ml:200 ml:30 g:2.5 ml:20 ml; and

ii. preparation of a porous zirconium dioxide nano microsphere: adding the polystyrene microsphere powders obtained in step i into the solution A obtained in step (2), introducing nitrogen, adding the zirconium dioxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, carrying out suction filtration to obtain a white precipitate, then washing the white precipitate with absolute ethyl alcohol, and drying to form the porous zirconium dioxide nano microsphere; preferably, the drying temperature being 100° C.; the drying time being 4 h; a weight-to-volume ratio of solution A to polystyrene microsphere powders to the zirconium dioxide precursor to N,N-dimethylformamide being 70 ml:2 g:30 ml:20 ml.

According to the extracellular vesicle purification material of embodiments of the present disclosure, further, a method for preparing the porous titanium dioxide nano microsphere comprises the following steps:

(1) preparation of a titanium dioxide precursor: dissolving tetrabutyl titanate into absolute ethyl alcohol, then adding deionized water, sufficiently and uniformly mixing, then adding concentrated hydrochloric acid, sealing, and then placing for 1-3 days at room temperature to obtain a light yellow transparent liquid, namely, titanium dioxide precursor; preferably, a volume ratio of tetrabutyl titanate to absolute ethyl alcohol to deionized water to concentrated hydrochloric acid being 4:2:50:0.7;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml;

i. preparation of polystyrene microsphere powders: adding a first polystyrene and a first ammonium persulfate into the solution A obtained in step (2), sufficiently stirring and emulsifying under the atmosphere of nitrogen, dropwise adding a second polystyrene when stirring and heating to 45-55° C., adding a second ammonium persulfate after completion of dropwise addition, heating to 80-90° C., continuing to stir under the atmosphere of nitrogen until being evenly mixed, cooling to 45-55° C., then dropwise adding a sodium chloride solution, carrying out suction filtration to obtain a white precipitate, then washing the white precipitate with absolute ethyl alcohol and deionized water in turn, and then drying to form the polystyrene microsphere powders; the concentrations of ammonium persulfate being both 200 mg/ml; the concentration of the sodium chloride solution being 2M; the drying temperature being 60° C.; a weight-to-volume ratio of the first polystyrene to the first ammonium persulfate to solution A to the second polystyrene to the second ammonium persulfate to the sodium chloride solution being 30 g:2.5 ml:200 ml:30 g:2.5 ml:20 ml; and

ii. preparation of a porous titanium dioxide nano microsphere: adding the polystyrene microsphere powders obtained in step i into the solution A obtained in step (2), introducing nitrogen, adding the titanium dioxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, carrying out suction filtration to obtain a white precipitate, then washing the white precipitate with absolute ethyl alcohol, and drying to form the porous titanium dioxide nano microsphere; preferably, the drying temperature being 100° C.; the drying time being 4 h; a weight-to-volume ratio of the solution A to the polystyrene microsphere powders to the zirconium dioxide precursor to N,N-dimethylformamide being 50 ml:2 g:50 ml:20 ml.

According to the extracellular vesicle purification material of embodiments of the present disclosure, further, a method for preparing the porous aluminum oxide nano microsphere comprises the following steps:

(1) preparation of an aluminum oxide precursor: dissolving sodium metaaluminate into deionized water, and sufficiently stirring until being completely dissolved to form a uniform and transparent solution, namely, the aluminum oxide precursor; preferably, a volume ratio of sodium metaaluminate to deionized water being 0.1 g:50 ml;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml;

i. preparation of polystyrene microsphere powders: adding a first polystyrene and a first ammonium persulfate into the solution A obtained in step (2), sufficiently stirring and emulsifying under the atmosphere of nitrogen, dropwise adding a second polystyrene when stirring and heating to 45-55° C., adding a second ammonium persulfate after completion of dropwise addition, heating to 80-90° C., continuing to stir under the atmosphere of nitrogen until being evenly mixed, cooling to 45-55° C., then dropwise adding a sodium chloride solution, carrying out suction filtration to obtain a white precipitate, then washing the white precipitate with absolute ethyl alcohol and deionized water in turn, and then drying to form the polystyrene microsphere powders; the concentrations of ammonium persulfate being both 200 mg/ml; the concentration of the sodium chloride solution being 2M; the drying temperature being 60° C.; a weight-to-volume ratio of the first polystyrene to the first ammonium persulfate to solution A to the second polystyrene to the second ammonium persulfate to the sodium chloride solution being 30 g:2.5 ml:200 ml:30 g:2.5 ml:20 ml; and

ii. preparation of a porous aluminum oxide nano microsphere: adding the polystyrene microsphere powders obtained in step i into the solution A obtained in step (2), introducing nitrogen, adding the aluminum oxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, carrying out suction filtration to obtain a white precipitate, then washing the white precipitate with absolute ethyl alcohol, and drying to form the porous aluminum oxide nano microsphere; preferably, the drying temperature being 100° C.; the drying time being 4 h; a weight-to-volume ratio of solution A to polystyrene microsphere powder to aluminum oxide precursor to N,N-dimethylformamide being 50 ml:2 g:50 ml:20 ml.

According to an extracellular vesicle purification method of embodiments of the present disclosure, the purification method comprises: purifying an extracellular vesicle using the purification material.

Preferably, a binding buffer and the purification material are added into a sample to be detected in sequence to be mixed and acted so that the extracellular vesicles are adsorbed in the purification material, and then the purification material is eluted with an eluent so as to obtain purified extracellular vesicles.

Specifically, after 500 μl of samples are mixed with 500 μl of binding buffer and then 100 μl of metal oxide microspheres or metal oxide magnetic beads are added to be gently mixed with the above obtained mixture, the metal oxide microspheres or metal oxide magnetic beads reversely bind to phosphatidylserine to act for 15 min at room temperature, so that the extracellular vesicles are adsorbed on the surfaces of microspheres or magnetic beads and other impurities are removed without adsorption, and the microspheres or magnetic beads adsorbed with the extracellular vesicles are then washed with normal saline or phosphate buffer or citric acid buffer, and finally washed with sodium acetate-sodium bicarbonate or 10% ammonia or 50 mmol/L trimethylaminomethane eluent, so as to obtain purified extracellular vesicles.

Specifically, the binding buffer is an acetic acid-sodium acetate solution, and the binding buffer is a 1M pH 5.5 acetic acid-sodium acetate solution.

Preferably, under the action of binding buffer, the microspheres or magnetic beads in the sample reversibly bind to phosphatidylserine, the extracellular vesicles are adsorbed on the surface of the microspheres or magnetic beads, other impurities are removed without adsorption, and then the microspheres or magnetic beads adsorbed with extracellular vesicles are washed with washing liquor, and finally washed with an eluant to obtain the purified extracellular vesicles; the eluent is one or a combination of sodium acetate sodium bicarbonate or 10% ammonia or 50 mmol/L tris(hydroxymethylaminomethane) eluent.

The lipid bilayer is composed of amphiphilic phospholipids with a hydrophobic tail and a hydrophilic phosphate head. In a biological system, the hydrophilic phosphate head of the phospholipid is exposed to the outer surface of the lipid bilayer. It is well known that some metal oxides, such as zirconium dioxide (ZrO₂), titanium oxide (TiO₂) and alumina (Al₂O₃), can reversibly bind to a phosphate group with high specificity. By using this characteristic, a metal oxide has been widely used in highly selective synthesis and enrichment of phosphorylated peptides, water-soluble organophosphorus and organophosphorus pesticides. In view of this, we tried to enrich extracellular vesicles by bidentate binding using micron-scale metal oxides. Due to its simple high affinity binding between phosphate groups and metal oxides on the surface of the lipid bilayer, this selective enrichment based on a solid phase has great advantages, for example the isolation efficiency of EVs is improved, non-specific protein adsorption is reduced and the sample processing time is shortened.

The principle of the purification method of the present disclosure: the extraction of extracellular vesicles by a capture method is based on the reversible binding and elution of phosphatidylserine (PS). The present disclosure adopts the purification method of microsphere particles/magnetic particles which have high affinity with extracellular vesicles. Under the action of binding buffer (EV-CB), the extracellular vesicles in the sample are specifically adsorbed on the surfaces of microspheres/magnetic beads, and other impurities such as non phospholipid proteins are removed without adsorption; the microspheres or magnetic beads adsorbed with the extracellular vesicles are washed with the washing liquor to remove proteins and impurities, and finally, the pure extracellular vesicles are eluted by eluent EB.

Compared with the prior art, the present disclosure has the following beneficial effects:

(1) The purification method of the present disclosure can perform quick extraction: the whole purification process is shortened within 1 h (microsphere method), or 30 min (magnetic bead method);

(2) The purification method of the present disclosure is more simple and convenient to operate: a simple centrifugation (or magnetic absorption)/mixing operation is only needed;

(3) The purification method of the present disclosure can purify multiple samples: serum/plasma, urine and cell culture supernatant;

(4) The purification method of the present disclosure has a moderate flux: it is suitable for rapid purification of extracellular vesicles with a medium sample size, high-throughput automatic extraction of sample extracellular vesicles can be realized after the magnetic bead method is adapted to an automatic extractor and corresponding operating procedures;

(5) The purification material microspheres or magnetic balls of the present disclosure are adopted, which means that a carrier is more flexible; the purification material microspheres or magnetic balls of the present disclosure can directly and specifically bind to phosphatidylserine (PS) so as to retain the extracellular vesicles to the greatest extent, high-purity extracellular vesicles can be obtained by elution and do not contain reagents that may affect subsequent experiments, such as a chelating agent.

### BRIEF DESCRIPTION OF THE DRAWINGS

For more clearly illustrating the technical solution in the embodiments of the present disclosure or in the prior art, drawings required for use in the embodiments or description of the prior art will be simply discussed below. Obviously, the drawings described below are only some embodiments of the present disclosure, and those of ordinary skill in the art can obtain other drawings according to these drawings without creative efforts.

FIG. 1a is a scanning electron micrograph (SEM) of cell culture supernatant extracellular vesicles purified by zirconium dioxide nano microspheres of the present disclosure;

FIG. 1b is an SEM of cell culture supernatant extracellular vesicles purified by titanium dioxide nano microspheres of the present disclosure;

FIG. 1c is an SEM of cell culture supernatant extracellular vesicles purified by aluminum oxide nano microspheres of the present disclosure;

FIG. 2 is a nanoparticle tracking analysis (NTA) identification graph of cell culture supernatant extracellular vesicles purified by non-magnetic nano microspheres of the present disclosure;

FIG. 3 is a flow cytometry identification graph of cell culture supernatant extracellular vesicles purified by non-magnetic nano microspheres of the present disclosure;

FIG. 4a is an SEM of milk-derived extracellular vesicles purified by zirconium dioxide nano microspheres of the present disclosure;

FIG. 4b is an SEM of milk-derived extracellular vesicles purified by titanium dioxide nano microspheres of the present disclosure;

FIG. 4c is an SEM of milk-derived extracellular vesicles purified by aluminum oxide nano microspheres of the present disclosure;

FIG. 5 is a flow cytometry identification graph of milk-derived extracellular vesicles purified by non-magnetic nano microspheres of the present disclosure;

FIG. 6a is an SEM of human urine-derived extracellular vesicles purified by zirconium dioxide nano microspheres of the present disclosure;

FIG. 6b is an SEM of human urine-derived extracellular vesicles purified by titanium dioxide nano microspheres of the present disclosure;

FIG. 6c is an SEM of human urine-derived extracellular vesicles purified by aluminum oxide nano microspheres of the present disclosure;

FIG. 7 is a flow cytometry identification graph of human urine-derived extracellular vesicles purified by non-magnetic nano microspheres of the present disclosure;

FIG. 8 is a flow cytometric comparison and identification graph of yields of cell supernatant extracellular vesicles purified by different metal oxide magnetic beads of the present disclosure;

FIG. 9 is an SEM of human urine extracellular vesicles purified by using nano porous microspheres of the present disclosure as fillers; and

FIG. 10 is a carboxyfluorescein succinimidyl amino ester (CSFE) staining flow cytometry identification graph of human urine extracellular vesicles purified by using nano porous microspheres as fillers.

### DETAILED DESCRIPTION OF THE EMBODIMENTS

To make the objective, technical solution and advantages of the present disclosure more clear, the technical solution of the present disclosure will be described in detail below. Obviously, the described embodiments are only a part of embodiments of the present disclosure but not all the embodiments. Based on the embodiments of the present disclosure, other embodiments obtained by those of ordinary skill in the art without creative efforts are all included within the protective scope of the present disclosure.

In some specific embodiments, the extracellular vesicle purification material is a metal oxide microsphere or metal oxide magnetic bead that can reversibly bind to phosphatidylserine.

Specifically, the metal oxide microsphere is a non-magnetic nano microsphere or porous nano microsphere of zirconium dioxide, titanium dioxide or aluminum oxide, and the metal oxide magnetic bead is a magnetic nano microsphere of zirconium dioxide, titanium dioxide or aluminum oxide.

Further, the non-magnetic nano microsphere is a nano zirconium dioxide microsphere, a nano titanium dioxide microsphere or a nano aluminum oxide microsphere, the magnetic nano microsphere is a nano zirconium dioxide magnetic microsphere, a nano titanium dioxide magnetic microsphere or a nano aluminum oxide magnetic microsphere, and the nano porous microsphere is a porous zirconium dioxide nano microsphere, a porous titanium dioxide nano microsphere or a porous aluminum oxide nano microsphere.

The extracellular vesicle purification method comprises: purifying extracellular vesicles by using the metal oxide microspheres or metal oxide magnetic beads.

Preferably, under the action of binding buffer, the microspheres or magnetic beads in the sample reversibly bind to phosphatidylserine, the extracellular vesicles are adsorbed on the surface of the microspheres or magnetic beads, other impurities are removed without adsorption, and then the microspheres or magnetic beads adsorbed with extracellular vesicles are washed with washing liquor, and finally washed with an eluant to obtain the purified extracellular vesicles; the eluent is an EB eluent.

Specifically, a method for preparing the nano zirconium dioxide microsphere comprises the following steps:

(1) preparation of a zirconium dioxide precursor: mixing and heating zirconium dioxide powders, a first concentrated sulfuric acid and ammonium sulfate until being dissolved, adding a second concentrated sulfuric acid after the obtained mixture is placed at room temperature, uniformly mixing to form the zirconium dioxide precursor; a weight-to-volume ratio of the zirconium dioxide powders to the first concentrated sulfuric acid and the second concentrated sulfuric acid being 0.09-0.11 g:9-11 ml:2-4 g:18-22 ml;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml; and

(3) preparation of a nano zirconium dioxide microsphere: introducing nitrogen into the solution A obtained in step (2), adding the zirconium dioxide precursor obtained in step (1) under the state of stirring, then dropwise adding N,N-dimethylformamide, carrying out suction filtration after completion of reaction to obtain a white precipitate, washing the white precipitate with absolute ethyl alcohol, and then drying to obtain the nano zirconium dioxide microsphere; preferably, a volume ratio of the solution A to the zirconium dioxide precursor to N,N-dimethylformamide being 7:3:2.

Specifically, a method for preparing the nano titanium dioxide microsphere comprises the following steps:

(1) preparation of a titanium dioxide precursor: dissolving tetrabutyl titanate into absolute ethyl alcohol, then adding deionized water, sufficiently and uniformly mixing, then adding concentrated hydrochloric acid, sealing, and then placing for 1-3 days at room temperature to obtain a light yellow transparent liquid, namely, titanium dioxide precursor; preferably, a volume ratio of tetrabutyl titanate to absolute ethyl alcohol to deionized water to concentrated hydrochloric acid being 4:2:50:0.7;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml; and

(3) preparation of a nano titanium dioxide microsphere: introducing nitrogen into the solution A obtained in step (2), adding the titanium dioxide precursor obtained in step (1) under the state of stirring, then dropwise adding N,N-dimethylformamide, carrying out suction filtration after completion of reaction to obtain a white precipitate, washing with absolute ethyl alcohol and then drying to obtain the nano titanium dioxide microsphere; preferably, a volume ratio of the solution A to the titanium dioxide precursor to N,N-dimethylformamide being 7:3:2.

Specifically, a method for preparing the nano aluminum oxide magnetic microsphere comprises the following steps: 1001021(1) preparation of an aluminum oxide precursor: dissolving sodium metaaluminate into deionized water, and sufficiently stirring until being completely dissolved to form a uniform and transparent solution, namely, the aluminum oxide precursor; preferably, a volume ratio of sodium metaaluminate to deionized water being 0.1 g:50 ml;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml; and

(3) preparation of nano aluminum oxide microsphere: introducing nitrogen into the solution A obtained in step (2), adding the aluminum oxide precursor obtained in step (1) under the state of stirring, then dropwise adding N,N-dimethylformamide, carrying out suction filtration after completion of reaction to obtain a white precipitate, washing with absolute ethyl alcohol and then drying to obtain the nano aluminum oxide microsphere; preferably, a volume ratio of the solution A to the aluminum oxide precursor to N,N-dimethylformamide being 7:3:2.

Specifically, a method for preparing the nano zirconium dioxide magnetic microsphere comprises the following steps:

(1) preparation of a zirconium dioxide precursor: mixing and heating zirconium dioxide powders, a first concentrated sulfuric acid and ammonium sulfate until being dissolved, adding a second concentrated sulfuric acid after the obtained mixture is placed at room temperature, uniformly mixing to form the zirconium dioxide precursor; preferably, a weight-to-volume ratio of the zirconium dioxide powders to the first concentrated sulfuric acid and the second concentrated sulfuric acid being 0.09-0.11 g:9-11 ml:2-4 g:18-22 ml;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml;

I. preparation of ferroferric oxide microsphere powders: dissolving ferric trichloride and ferrous sulfate into the solution A obtained in step (2), sufficiently stirring until being evenly mixed, dropwise adding a saturated ammonium bicarbonate solution under the state of stirring, meanwhile introducing nitrogen, stirring and heating, dropwise adding sodium hydroxide solution when the temperature is raised to 75-85° C., continuing to stir and mix under the atmosphere of nitrogen after observing that the solution turns pure black, harvesting a black precipitate via magnetic absorption, washing the black precipitate with absolute ethyl alcohol and deionized water in turn, and then drying to form the ferroferric oxide microsphere powders; preferably, the drying temperature being 60° C.; the concentration of the sodium hydroxide solution being 1M; a weight-to-volume ratio of ferric trichloride to ferrous sulfate to saturated ammonium bicarbonate solution to 1M sodium hydroxide solution being 3.24 g:2.78 g:50 ml:10 ml; and

II. preparation of a nano zirconium dioxide magnetic microsphere: adding the ferroferric oxide microsphere powders obtained in step I into the solution A obtained in step (2), introducing nitrogen, adding the zirconium dioxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, magnetically absorbing a brown precipitate, then washing the brown precipitate with absolute ethyl alcohol, and drying to obtain the nano zirconium dioxide magnetic microsphere; preferably, the drying temperature being 100° C.; the drying time being 4 h; a weight-to-volume ratio of the solution A to the ferroferric oxide microsphere powder to the zirconium dioxide precursor to N,N-dimethylformamide being 70 ml:2 g:30 ml:20 ml.

Specifically, a method for preparing the nano titanium dioxide magnetic microsphere comprises the following steps:

(1) preparation of a titanium dioxide precursor: dissolving tetrabutyl titanate into absolute ethyl alcohol, then adding deionized water, sufficiently and uniformly mixing, then adding concentrated hydrochloric acid, sealing, and then placing for 1-3 days at room temperature to obtain a light yellow transparent liquid, namely, titanium dioxide precursor; preferably, a volume ratio of tetrabutyl titanate to absolute ethyl alcohol to deionized water to concentrated hydrochloric acid being 4:2:50:0.7;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml;

I. preparation of ferroferric oxide microsphere powders: dissolving ferric trichloride and ferrous sulfate into the solution A obtained in step (2), sufficiently stirring until being evenly mixed, dropwise adding a saturated ammonium bicarbonate solution under the state of stirring, meanwhile introducing nitrogen, stirring and heating, dropwise adding a sodium hydroxide solution when a temperature is raised to 75-85° C., continuing to stir and mix under the atmosphere of nitrogen after observing that the solution turns pure black, harvesting a black precipitate via magnetic absorption, washing the black precipitate with absolute ethyl alcohol and deionized water in turn, and then drying to form the ferroferric oxide microsphere powders; preferably, the drying temperature being 60° C.; the concentration of the sodium hydroxide solution being 1M; a weight-to-volume ratio of ferric trichloride to ferrous sulfate to saturated ammonium bicarbonate solution to 1M sodium hydroxide solution being 3.24 g:2.78 g:50 ml:10 ml; and

II. preparation of a nano titanium dioxide magnetic microsphere: adding the ferroferric oxide microsphere powders obtained in step I into the solution A obtained in step (2), introducing nitrogen, adding the titanium dioxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, magnetically absorbing a brown precipitate, then washing the brown precipitate with absolute ethyl alcohol, and drying to obtain the nano titanium dioxide magnetic microsphere; preferably, the drying temperature being 100° C.; the drying time being 4 h; a weight-to-volume ratio of the solution A to the ferroferric oxide microsphere powder to the titanium dioxide precursor to N,N-dimethylformamide being 50 ml:2 g:50 ml:20 ml.

Specifically, a method for preparing the nano aluminum oxide magnetic microsphere comprises the following steps:

(1) preparation of an aluminum oxide precursor: dissolving sodium metaaluminate into deionized water, and sufficiently stirring until being completely dissolved to form a uniform and transparent solution, namely, the aluminum oxide precursor; preferably, a volume ratio of sodium metaaluminate to deionized water being 0.1 g:50 ml;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml;

I. preparation of ferroferric oxide microsphere powders: dissolving ferric trichloride and ferrous sulfate into the solution A obtained in step (2), sufficiently stirring until being evenly mixed, dropwise adding a saturated ammonium bicarbonate solution under the state of stirring, meanwhile introducing nitrogen, stirring and heating, dropwise adding a sodium hydroxide solution when a temperature is raised to 75-85° C., continuing to stir and mix under the atmosphere of nitrogen after observing that the solution turns pure black, harvesting a black precipitate via magnetic absorption, washing the black precipitate with absolute ethyl alcohol and deionized water in turn, and then drying to form the ferroferric oxide microsphere powders; preferably, the drying temperature being 60° C.; the concentration of the sodium hydroxide solution being 1M; a weight-to-volume ratio of ferric trichloride to ferrous sulfate to saturated ammonium bicarbonate solution to 1M sodium hydroxide solution being 3.24 g:2.78 g:50 ml:10 ml; and

II. preparation of a nano aluminum oxide magnetic microsphere: adding the ferroferric oxide microsphere powders obtained in step I into the solution A obtained in step (2), introducing nitrogen, adding the aluminum oxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, magnetically absorbing a brown precipitate, then washing the brown precipitate with absolute ethyl alcohol, and drying to obtain the nano aluminum oxide magnetic microsphere; preferably, the drying temperature being 100° C.; the drying time being 4 h; a weight-to-volume ratio of the solution A to the ferroferric oxide microsphere powder to the aluminum oxide precursor to N,N-dimethylformamide being 50 ml:2 g:50 ml:20 ml.

Specifically, a method for preparing the porous zirconium dioxide nano microsphere comprises the following steps:

(1) preparation of a zirconium dioxide precursor: mixing and heating zirconium dioxide powders, a first concentrated sulfuric acid and ammonium sulfate until being dissolved, adding a second concentrated sulfuric acid after the obtained mixture is placed at room temperature, uniformly mixing to form the zirconium dioxide precursor; preferably, a weight-to-volume ratio of the zirconium dioxide powders to the first concentrated sulfuric acid and the second concentrated sulfuric acid being 0.09-0.11 g:9-11 ml:2-4 g:18-22 ml;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml;

i. preparation of polystyrene microsphere powders: adding a first polystyrene and a first ammonium persulfate into the solution A obtained in step (2), sufficiently stirring and emulsifying under the atmosphere of nitrogen, dropwise adding a second polystyrene when stirring and heating to 45-55° C., adding a second ammonium persulfate after completion of dropwise addition, heating to 80-90° C., continuing to stir under the atmosphere of nitrogen until being evenly mixed, cooling to 45-55° C., then dropwise adding a sodium chloride solution, carrying out suction filtration to obtain a white precipitate, then washing the white precipitate with absolute ethyl alcohol and deionized water in turn, and then drying to form the polystyrene microsphere powders; the concentrations of ammonium persulfate being both 200 mg/ml; the concentration of the sodium chloride solution being 2M; the drying temperature being 60° C.; a weight-to-volume ratio of the first polystyrene to the first ammonium persulfate to solution A to the second polystyrene to the second ammonium persulfate to the sodium chloride solution being 30 g:2.5 ml:200 ml:30 g:2.5 ml:20 ml; and

ii. preparation of a porous zirconium dioxide nano microsphere: adding the polystyrene microsphere powders obtained in step i into the solution A obtained in step (2), introducing nitrogen, adding the zirconium dioxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, carrying out suction filtration to obtain a white precipitate, then washing the white precipitate with absolute ethyl alcohol, and drying to form the porous zirconium dioxide nano microsphere; preferably, the drying temperature being 100° C.; the drying time being 4 h; a weight-to-volume ratio of solution A to polystyrene microsphere powders to the zirconium dioxide precursor to N,N-dimethylformamide being 70 ml:2 g:30 ml:20 ml.

Specifically, a method for preparing the porous titanium dioxide nano microsphere comprises the following steps:

(1) preparation of a titanium dioxide precursor: dissolving tetrabutyl titanate into absolute ethyl alcohol, then adding deionized water, sufficiently and uniformly mixing, then adding concentrated hydrochloric acid, sealing, and then placing for 1-3 days at room temperature to obtain a light yellow transparent liquid, namely, titanium dioxide precursor; preferably, a volume ratio of tetrabutyl titanate to absolute ethyl alcohol to deionized water to concentrated hydrochloric acid being 4:2:50:0.7;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml;

i. preparation of polystyrene microsphere powders: adding a first polystyrene and a first ammonium persulfate into the solution A obtained in step (2), sufficiently stirring and emulsifying under the atmosphere of nitrogen, dropwise adding a second polystyrene when stirring and heating to 45-55° C., adding a second ammonium persulfate after completion of dropwise addition, heating to 80-90° C., continuing to stir under the atmosphere of nitrogen until being evenly mixed, cooling to 45-55° C., then dropwise adding a sodium chloride solution, carrying out suction filtration to obtain a white precipitate, then washing the white precipitate with absolute ethyl alcohol and deionized water in turn, and then drying to form the polystyrene microsphere powders; the concentrations of ammonium persulfate being both 200 mg/ml; the concentration of the sodium chloride solution being 2M; the drying temperature being 60° C.; a weight-to-volume ratio of the first polystyrene to the first ammonium persulfate to solution A to the second polystyrene to the second ammonium persulfate to the sodium chloride solution being 30 g:2.5 ml:200 ml:30 g:2.5 ml:20 ml; and

ii. preparation of a porous titanium dioxide nano microsphere: adding the polystyrene microsphere powders obtained in step i into the solution A obtained in step (2), introducing nitrogen, adding the titanium dioxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, carrying out suction filtration to obtain a white precipitate, then washing the white precipitate with absolute ethyl alcohol, and drying to form the porous titanium dioxide nano microsphere; preferably, the drying temperature being 100° C.; the drying time being 4 h; a weight-to-volume ratio of the solution A to the polystyrene microsphere powders to the zirconium dioxide precursor to N,N-dimethylformamide being 50 ml:2 g:50 ml:20 ml.

Specifically, a method for preparing the porous aluminum oxide nano microsphere comprises the following steps:

(1) preparation of an aluminum oxide precursor: dissolving sodium metaaluminate into deionized water, and sufficiently stirring until being completely dissolved to form a uniform and transparent solution, namely, the aluminum oxide precursor; preferably, a volume ratio of sodium metaaluminate to deionized water being 0.1 g:50 ml;

(2) preparation of solution A: dissolving povidone and sodium dodecylbenzenesulfonate into deionized water to be sufficiently stirred and dissolved, so as to form the solution A; preferably, a weight-to-volume ratio of povidone to sodium dodecylbenzenesulfonate to deionized water being 0.05 g:2 g:100 ml;

i. preparation of polystyrene microsphere powders: adding a first polystyrene and a first ammonium persulfate into the solution A obtained in step (2), sufficiently stirring and emulsifying under the atmosphere of nitrogen, dropwise adding a second polystyrene when stirring and heating to 45-55° C., adding a second ammonium persulfate after completion of dropwise addition, heating to 80-90° C., continuing to stir under the atmosphere of nitrogen until being evenly mixed, cooling to 45-55° C., then dropwise adding a sodium chloride solution, carrying out suction filtration to obtain a white precipitate, then washing the white precipitate with absolute ethyl alcohol and deionized water in turn, and then drying to form the polystyrene microsphere powders; the concentrations of ammonium persulfate being both 200 mg/ml; the concentration of the sodium chloride solution being 2M; the drying temperature being 60° C.; a weight-to-volume ratio of the first polystyrene to the first ammonium persulfate to solution A to the second polystyrene to the second ammonium persulfate to the sodium chloride solution being 30 g:2.5 ml:200 ml:30 g:2.5 ml:20 ml; and

ii. preparation of a porous aluminum oxide nano microsphere: adding the polystyrene microsphere powders obtained in step i into the solution A obtained in step (2), introducing nitrogen, adding the aluminum oxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, carrying out suction filtration to obtain a white precipitate, then washing the white precipitate with absolute ethyl alcohol, and drying to form the porous aluminum oxide nano microsphere; preferably, the drying temperature being 100° C.; the drying time being 4 h; a weight-to-volume ratio of solution A to polystyrene microsphere powder to aluminum oxide precursor to N,N-dimethylformamide being 50 ml:2 g:50 ml:20 ml.

Next, the technical solution of the present disclosure will be described in detail through examples in combination with drawings. However, the selected examples are only for illustrating the present disclosure but not limiting the scope of the present disclosure.

### Example 1

This example provides an extracellular vesicle purification method: extracellular vesicles were purified by using nano zirconium dioxide microspheres;

a method for preparing the nano zirconium dioxide microsphere comprises the following steps:

(1) self-preparation of a zirconium dioxide precursor: 0.10 g of zirconium dioxide powders were accurately weighed and put into a 150 mL beaker, 10 mL of concentrated sulfuric acid and 3 g of ammonium sulfate were added, the beaker was covered with a watch glass and heated on an electric furnace so that the above materials were dissolved; after the sample was completely dissolved, the beaker was taken out and slightly cooled, then the dissolved sample was put into a 200 mL volumetric flask, and then 20 ml of concentrated sulfuric acid was added;

(2) solution A: 0.05 g of povidone and 2 g of sodium dodecylbenzenesulfonate were added into 100 ml of deionized water to be fully stirred and dissolved for standby;

(3) preparation of a nano zirconium dioxide microsphere: 70 ml of solution A was added into a stirring bottle, nitrogen was introduced into the bottle, the solution A was stirred for 15 min at 30° C. and 800 rpm, 30 ml of zirconium dioxide precursor was added slowly under the state of stirring, the above materials were continued to be stirred for 1 h, 20 ml of N,N-dimethylformamide was added into the obtained mixed solution at a speed of one drop every 3 seconds, the resulting mixed solution was heated to 40° C. after completion of dropwise addition and then continued to be stirred for 12 h under the atmosphere of nitrogen at 800 rpm, suction filtration was carried out to obtain a white precipitate, and the white precipitate was washed three times with absolute ethyl alcohol and dried for 2 h in a muffle furnace at 600° C. to obtain the nano zirconium dioxide microsphere.

### Example 2

This example provides an extracellular vesicle purification method: a sample was put into a buffer, and then nano titanium dioxide microspheres that can reversely bind to phosphatidtlserine were put so that extracellular vesicles were adsorbed on the surfaces of the microspheres and other impurities were removed without adsorption, and then the microspheres adsorbed with the extracellular vesicles were washed with a washing liquor and finally washed with an EB eluent to obtain purified extracellular vesicles;

a method for preparing the nano titanium dioxide microsphere comprises the following steps:

(1) self-preparation of a titanium dioxide precursor: 8 ml of tetrabutyl titanate was dissolved into 4 ml of absolute ethyl alcohol, 100 ml of deionized water was slowly added, the above materials were fully stirred, then 1.4 ml of concentrated hydrochloric acid was added, and the resulting mixture was placed for 48 h at room temperature after being sealed to obtain a light yellow transparent liquid;

(2) solution A: 0.05 g of povidone and 2 g of sodium dodecylbenzenesulfonate were added into 100 ml of deionized water to be fully stirred and dissolved for standby;

(3) preparation of a nano titanium dioxide microsphere: 50 ml of solution A was added into a stirring bottle, nitrogen was introduced into the bottle, the solution A was stirred for 15 min at 30° C. and 800 rpm, 50 ml of titanium dioxide precursor was slowly added under the state of stirring, the above materials were continued to be stirred for 1 h, 20 ml of N,N-dimethylformamide was added into the mixed solution at a speed of one drop every 3 seconds, the resulting mixed solution was heated to 40° C. after completion of dropwise addition and then continued to be stirred for 12 h under the atmosphere of nitrogen at 800 rpm, suction filtration was carried out to obtain a white precipitate, and then the white precipitate was washed three times with absolute ethyl alcohol and dried for 2 h in a muffle furnace at 600° C. to obtain the nano titanium dioxide microspheres.

### Example 3

This example provides an extracellular vesicle purification method: a sample was put into a buffer, and then nano aluminum oxide microspheres that can reversely bind to phosphatidtlserine were put so that extracellular vesicles were adsorbed on the surfaces of the microspheres and other impurities were removed without adsorption, and then the microspheres adsorbed with the extracellular vesicles were washed with a washing liquor and finally washed with an EB eluent to obtain purified extracellular vesicles;

a method for preparing the nano aluminum oxide microsphere comprises the following steps:

(1) self-preparation of an aluminum oxide precursor: 0.1 g of sodium metaaluminate was added into 50 ml of deionized water to be fully stirred and dissolved to prepare a homogeneous transparent liquid;

(2) solution A: 0.05 g of povidone and 2 g of sodium dodecylbenzenesulfonate were added into 100 ml of deionized water to be fully stirred and dissolved for standby;

(3) preparation of a nano aluminum oxide microsphere: 50 ml of solution A was added into a stirring bottle, nitrogen was introduced into the bottle, the above materials were stirred for 15 min at 30° C. and 800 rpm, 50 ml of aluminum oxide precursor was slowly added under the state of stirring, the above materials were continued to be stirred for 1 h, 20 ml of N,N-dimethylformamide was added into the mixed solution at a speed of one drop every 3 seconds, the resulting mixed solution was heated to 40° C. and then continued to be stirred for 12 h at 40° C. under the atmosphere of nitrogen at 800 rpm, suction filtration was carried out to obtain a white precipitate, and then the white precipitate was washed three times with absolute ethyl alcohol and dried for 2 h in a muffle furnace at 600° C. to obtain the nano aluminum oxide microsphere.

### Example 4

This example provides an extracellular vesicle purification method: a sample was put into a buffer, then nano zirconium dioxide microspheres (magnetic spheres) that can reversely bind to phosphatidtlserine were put so that extracellular vesicles were adsorbed on the surfaces of the microspheres and other impurities were removed without adsorption, and then the microspheres adsorbed with the extracellular vesicles were washed with a washing liquor and finally washed with an EB eluent to obtain purified extracellular vesicles;

a method for preparing the nano zirconium dioxide magnetic microsphere comprises the following steps:

(1) self-preparation of a zirconium dioxide precursor: 0.1 g of zirconium dioxide powders were accurately weighed and put into a 150 mL beaker, 10 mL of concentrated sulfuric acid and 3 g of ammonium sulfate were added, the beaker was covered with a watch glass and then put on an electric furnace to be heated so that the above materials were dissolved; after the sample was completely dissolved, the beaker was taken out and slightly cooled, then the dissolved sample was put into a 200 mL volumetric flask, and then 20 mL of concentrated sulfuric acid was added;

(2) solution A: 0.05 g of povidone and 2 g of sodium dodecylbenzenesulfonate were added into 100 ml of deionized water to be fully stirred and dissolved for standby;

(3) preparation of a ferroferric oxide microsphere: 3.24 g of ferric trichloride and 2.78 g of ferrous sulfate were dissolved into 100 ml of solution A and fully stirred, then the volume was metered to 200 ml with the solution A, the resulting solution was stirred for 15 min at 30° C. and 800 rpm, 50 ml of saturated ammonium bicarbonate solution was dropwise added under the state of stirring and meanwhile nitrogen was introduced, the resulting mixed solution was stirred and heated, 10 ml of 1M sodium hydroxide solution was dropwise added when the temperature was raised to 80° C., the above mixed solution was continued to be stirred for 2 h under the atmosphere of nitrogen after being observed to turned pure black, and a black precipitate was harvested via magnetic absorption, washed three times with absolute ethyl alcohol, washed three times with deionized water, and then dried for 12 h in an oven at 60° C.;

(4) preparation of a nano zirconium dioxide magnetic microsphere: 70 ml of solution A was added into a stirring bottle, 2 g of ferroferric oxide microsphere powders were accurately weighed, nitrogen was introduced into the bottle, the above materials were stirred for 15 min at 30° C. and 800 rpm, 30 ml of zirconium dioxide precursor was slowly added under the state of stirring, the above materials were continued to be stirred for 1 h, 20 ml of N,N-dimethylformamide was added into the mixed solution at a speed of one drop every 3 seconds, the resulting mixed solution was heated after completion of dropwise addition and continued to be stirred for 12 h at 40° C. under the atmosphere of nitrogen at 800 rpm, a brown precipitate was harvested via magnetic absorption, then washed three times with absolute ethyl alcohol and dried for 4 h in a muffle furnace at 600° C. to obtain the nano zirconium dioxide microsphere.

### Example 5

This example provides an extracellular vesicle purification method: a sample was put into a buffer, and then nano titanium dioxide microspheres (magnetic spheres) that can reversely bind to phosphatidtlserine were put so that extracellular vesicles were adsorbed on the surfaces of the microspheres and other impurities were removed without adsorption, and then the microspheres adsorbed with the extracellular vesicles were washed with a washing liquor and finally washed with an EB eluent to obtain purified extracellular vesicles;

a method for preparing the nano titanium dioxide microsphere comprises the following steps:

(1) self-preparation of a titanium dioxide precursor: 8 ml of tetrabutyl titanate was dissolved into 4 ml of absolute ethyl alcohol, 100 ml of deionzied water was slowly added, the above materials were stirred, then 1.4 ml of concentrated hydrochloric acid was added, and the resulting mixture was placed for 48 h at room temperature after being sealed to obtain a light yellow transparent liquid;

(2) solution A: 0.05 g of povidone and 2 g of sodium dodecylbenzenesulfonate were added into 100 ml of deionized water to be fully stirred and dissolved for standby;

(3) preparation of a ferroferric oxide microsphere: 3.24 g of ferric trichloride and 2.78 g of ferrous sulfate were dissolved into 100 ml of solution A and fully stirred, then the volume was metered to 200 ml with the solution A, the resulting solution was stirred for 15 min at 30° C. and 800 rpm, 50 ml of saturated ammonium bicarbonate solution was dropwise added under the state of stirring and meanwhile nitrogen was introduced, the above mixed solution was stirred and heated, 10 ml of 1M sodium hydroxide solution was dropwise added when the temperature was raised to 80° C., introduction of nitrogen and stirring were simultaneously performed after the solution was observed to turned pure black, and a black precipitate was harvested via magnetic absorption, washed three times with absolute ethyl alcohol, washed three times with deionized water, and then dried for 12 h in an oven at 60° C.;

(4) preparation of a nano titanium dioxide magnetic microsphere: 50 ml of solution A was added into a stirring bottle, 2 g of ferroferric oxide microsphere powders were accurately weighed, nitrogen was introduced into the bottle, the above materials were stirred for 15 min at 30° C. and 800 rpm, 30 ml of titanium dioxide precursor was slowly added under the state of stirring, the above materials were continued to be stirred for 1 h, 20 ml of N,N-dimethylformamide was added into the mixed solution at a speed of one drop every 3 seconds, the mixed solution was heated to 40° C. after completion of dropwise addition and continued to be stirred for 12 h under the atmosphere of nitrogen at 800 rpm, and a brown precipitate was harvested by magnetic absorption, then washed three times with absolute ethyl alcohol and dried for 4 h in a muffle furnace at 600° C. to obtain the nano zirconium dioxide microsphere.

### Example 6

This example provides an extracellular vesicle purification method: a sample was put into a buffer, and then nano aluminum oxide magnetic microspheres (magnetic spheres) that can reversely bind to phosphatidtlserine were put so that extracellular vesicles were adsorbed on the surfaces of the microspheres and other impurities were removed without adsorption, and then the microspheres adsorbed with the extracellular vesicles were washed with a washing liquor and finally washed with an EB eluent to obtain purified extracellular vesicles;

a method for preparing the nano aluminum oxide magnetic microsphere comprises the following steps:

(1) self-preparation of an aluminum oxide precursor: 0.1 g of sodium metaaluminate was added into 50 ml of deionzied water at room temperature to be fully stirred and dissolved to prepare a homogenous transparent liquid;

(2) solution A: 0.05 g of povidone and 2 g of sodium dodecylbenzenesulfonate were added into 100 ml of deionized water to be fully stirred and dissolved for standby;

(3) preparation of a ferroferric oxide microsphere: 3.24 g of ferric trichloride and 2.78 g of ferrous sulfate were dissolved into 100 ml of solution A and fully stirred, the volume was metered to 200 ml with the solution A, the resulting solution was stirred for 15 min at 30° C. and 800 rpm, 50 ml of saturated ammonium bicarbonate solution was dropwise added under the state of stirring and meanwhile nitrogen was introduced, the resulting mixed solution was stirred and heated, 10 ml of 1M sodium hydroxide solution was dropwise added when the temperature was raised to 80° C., the above mixed solution was continued to be stirred for 2 h under the atmosphere of nitrogen after being observed to turned pure black, and a black precipitate was harvested via magnetic absorption, washed three times with absolute ethyl alcohol, washed three times with deionized water, and then dried for 12 h in an oven at 60° C.;

(4) preparation of a nano aluminum oxide magnetic microsphere: 50 ml of solution A was added into a stirring bottle, 2 g of ferroferric oxide microsphere powders were accurately weighed, nitrogen was introduced into the bottle, the above materials were stirred for 15 min at 30° C. and 800 rpm, 30 ml of aluminum oxide precursor was slowly added under the state of stirring, the above materials were continued to be stirred for 1 h, 20 ml of N,N-dimethylformamide was added into the mixed solution at a speed of one drop every 3 seconds, the mixed solution was heated to 40° C. after completion of dropwise addition and continued to be stirred for 12 h under the atmosphere of nitrogen at 800 rpm, and a brown precipitate was harvested by magnetic absorption, then washed three times with absolute ethyl alcohol and dried for 4 h in a muffle furnace at 600° C. to obtain the nano zirconium dioxide microsphere.

### Example 7

This example provides an extracellular vesicle purification method: a sample was put into a buffer, and then porous zirconium dioxide nano microspheres that can reversely bind to phosphatidtlserine were put so that extracellular vesicles were adsorbed on the surfaces of the microspheres and other impurities were removed without adsorption, and then the microspheres adsorbed with the extracellular vesicles were washed with a washing liquor and finally washed with an EB eluent to obtain purified extracellular vesicles;

a method for preparing the porous zirconium dioxide nano microsphere comprises the following steps:

(1) self-preparation of a zirconium dioxide precursor: 0.1 g of zirconium dioxide powder was accurately weighed and put into a 150 mL breaker, 10 mL of concentrated sulfuric acid and 3 g of ammonium sulfate were added, the beaker was covered with a watch glass and heated on an electric furnace so that the above materials were dissolved; after the sample was completely dissolved, the beaker was taken out and slightly cooled, then the dissolved sample was put into a 200 mL volumetric flask, and then 20 ml of concentrated sulfuric acid was added;

(2) solution A: 0.05 g of povidone and 2 g of sodium dodecylbenzenesulfonate were added into 100 ml of deionized water to be fully stirred and dissolved for standby;

(3) preparation of a polystyrene (PS) microsphere: 30 g of styrene and 2.5 ml of 200 mg/ml ammonium persulfate were accurately weighed and added into 200 ml of solution A stirred at room temperature at 1200 rpm, the above materials were fully stirred and emulsified for 4 h under the atmosphere of nitrogen and then heated to 50° C. at 1200 rpm under the atmosphere of nitrogen under the condition of stirring, the obtained mixture was added into 30 g of styrene mixed solution at a speed of one drop every 5 seconds, 2.5 ml of 200 mg/ml ammonium persulfate was added after completion of dropwise addition, the above materials were heated to 85° C. and then continued to be stirred for 2 h at 1200 rpm under the atmosphere of nitrogen and cooled to 50° C., then 20 ml of 2M sodium chloride solution was continued to be dropwise added, a white precipitate was obtained via suction filtration, washed three times with absolute ethyl alcohol, washed three times with deionized water and then dried for 12 h in an oven at 60° C.;

(4) preparation of a porous zirconium dioxide nano microsphere: 70 ml of solution A was added into a stirring bottle, 2 g of PS microsphere powders were accurately weighed, nitrogen was introduced into the bottle, the above materials were stirred for 15 min at 30° C. and 800 rpm, 30 ml of zirconium dioxide precursor was slowly added under the state of stirring, the above materials were continued to be stirred for 1 h, 20 ml of N,N-dimethylformamide was added into the mixed solution at a speed of one drop every 3 seconds, the mixed solution was heated to 40° C. after completion of dropwise addition and continued to be stirred for 12 h at 400 rpm under the atmosphere of nitrogen, and a white precipitate was obtained via suction filtration, washed three times with absolute ethyl alcohol and dried for 4 h in a muffle furnace at 600° C. to obtain the porous zirconium dioxide nano microsphere.

### Example 8

This example provides an extracellular vesicle purification method: a sample was put into a buffer, and then porous titanium dioxide nano microspheres that can reversely bind to phosphatidtlserine were put so that extracellular vesicles were adsorbed on the surfaces of the microspheres and other impurities were removed without adsorption, and then the microspheres adsorbed with the extracellular vesicles were washed with a washing liquor and finally washed with an EB eluent to obtain purified extracellular vesicles;

a method for preparing the porous titanium dioxide nano microsphere comprises the following steps:

(1) self-preparation of a titanium dioxide precursor: 8 ml of tetrabutyl titanate was dissolved into 4 ml of absolute ethyl alcohol, 100 ml of deionzied water was slowly added to be fully stirred, then 1.4 ml of concentrated hydrochloric acid was added, and the above mixture was placed for 48 h at room temperature after being sealed to obtain a light yellow transparent liquid;

(2) solution A: 0.05 g of povidone and 2 g of sodium dodecylbenzenesulfonate were added into 100 ml of deionized water to be fully stirred and dissolved for standby;

(3) preparation of a polystyrene (PS) microsphere: 30 g of styrene and 2.5 ml of 200 mg/ml ammonium persulfate were accurately weighed and added into 200 ml of solution A stirred at room temperature at 1200 rpm, the above materials were fully stirred and emulsified for 4 h under the atmosphere of nitrogen and then heated to 50° C. at 1200 rpm under the atmosphere of nitrogen under the condition of stirring, the above mixture was added into 30 g of styrene mixed solution at a speed of one drop every 5 seconds, 2.5 ml of 200 mg/ml ammonium persulfate was added after completion of dropwise addition, the above materials were heated to 85° C. and then continued to be stirred for 2 h at 1200 rpm under the atmosphere of nitrogen and cooled to 50° C., then 20 ml of 2M sodium chloride solution was continued to be added, a white precipitate was obtained via suction filtration, washed three times with absolute ethyl alcohol, washed three times with deionized water and then dried for 12 h in an oven at 60° C.;

(4) preparation of a porous titanium dioxide nano microsphere: 50 ml of solution A was added into a stirring bottle, 2 g of PS microsphere powders were accurately weighed, nitrogen was introduced into the bottle, the above materials were stirred for 15 min at 30° C. and 800 rpm, 30 ml of titanium dioxide precursor was slowly added under the state of stirring, the above materials were continued to be stirred for 1 h, 20 ml of N,N-dimethylformamide was added into the mixed solution at a speed of one drop every 3 seconds, the mixed solution was heated to 40° C. after completion of dropwise addition and continued to be stirred for 12 h at 400 rpm under the atmosphere of nitrogen, and a white precipitate was obtained via suction filtration, washed three times with absolute ethyl alcohol and dried for 4 h in a muffle furnace at 600° C. to obtain the porous titanium dioxide nano microsphere.

### Example 9

This example provides an extracellular vesicle purification method: a sample was put into a buffer, and then porous aluminum oxide nano microspheres that can reversely bind to phosphatidtlserine were put so that extracellular vesicles were adsorbed on the surfaces of the microspheres and other impurities were removed without adsorption, and then the microspheres adsorbed with the extracellular vesicles were washed with a washing liquor and finally washed with an EB eluent to obtain purified extracellular vesicles;

a method for preparing the porous aluminum oxide nano microsphere comprises the following steps:

(1) self-preparation of an aluminum oxide precursor: 0.1 g of sodium metaaluminate was dissolved into 50 ml of deionzied water to be fully stirred and dissolved to prepare a homogenous transparent liquid;

(2) solution A: 0.05 g of povidone and 2 g of sodium dodecylbenzenesulfonate were added into 100 ml of deionized water to be fully stirred and dissolved for standby;

(3) preparation of a polystyrene (PS) microsphere: 30 g of styrene and 2.5 ml of 200 mg/ml ammonium persulfate were accurately weighed and added into 200 ml of solution A stirred at room temperature at 1200 rpm, the above materials were fully stirred and emulsified for 4 h under the atmosphere of nitrogen and then heated to 50° C. at 1200 rpm under the atmosphere of nitrogen under the condition of stirring, the above mixture was added into 30 g of styrene mixed solution at a speed of one drop every 5 seconds, 2.5 ml of 200 mg/ml ammonium persulfate was added after completion of dropwise addition, the above materials were heated to 85° C. and then continued to be stirred for 2 h at 1200 rpm under the atmosphere of nitrogen and cooled to 50° C., then 20 ml of 2M sodium chloride solution was continued to be added, and a white precipitate was obtained via suction filtration, washed three times with absolute ethyl alcohol, washed three times with deionized water and then dried for 12 h in an oven at 60° C.;

(4) preparation of a porous aluminum oxide nano microsphere: 50 ml of solution A was added into a stirring bottle, 2 g of PS microsphere powders were accurately weighed, nitrogen was introduced into the bottle, the above materials were stirred for 15 min at 30° C. at 400 rpm, 50 ml of aluminum oxide precursor was slowly added under the state of stirring, the above materials were continued to be stirred for 1 h, 20 ml of N,N-dimethylformamide was added into the mixed solution at a speed of one drop every 3 seconds, the mixed solution was heated to 40° C. after completion of dropwise addition and continued to be stirred for 12 h at 400 rpm under the atmosphere of nitrogen, and a white precipitate was obtained via suction filtration, washed three times with absolute ethyl alcohol and dried for 4 h in a muffle furnace at 600° C. to obtain the porous zirconium dioxide nano microsphere.

Purification Effect Test

1. Purification of Cell Culture Supernatant Extracellular Vesicles with Non-Magnetic Nano Microshperes

Cell culture supernatant extracellular vesicles were purified by respectively using nano zirconium dioxide microspheres, nano titanium dioxide microspheres and nano aluminum oxide microspheres obtained in examples 1-3, which specifically comprises the following steps:

293T cell culture supernatant was freshly cultured and then centrifuged for 10 min at 3000 g to remove cell debris. 500 μl of centrifuged supernatant was put into a 1.5 ml EP tube, an equal volume of binding buffer (200 mM acetic acid-sodium acetate, pH 5.5) was added, 0.5% (M/V) non-magnetic nano microspheres were added based on weight, the above materials were gently and evenly mixed, the obtained mixture was placed for 15 min at room temperature and centrifuged for 5 min at 10000 g, supernatant was discarded, the microspheres were resuspended using 1 ml of washing liquor (0.02% OB-2 normal saline), the obtained suspension was centrifuged for 3 min at 10000 g, supernatant was discarded, and the microspheres were washed repeatedly twice; 5011.1 of elution A (100 mM sodium acetate-sodium bicarbonate, pH 10.0) was added to resuspend the microspheres, the resulting suspension was placed for 5 min at room temperature and centrifuged for 5 min at 10000 g, supernatant was transferred into a new 1.5 ml EP tube, 450 μl of elution B (normal saline) was added, the supernatant and the elution B were evenly mixed to obtain a product, that is, cell culture supernatant extracellular vesicles which can be directly used for subsequent experiments or cryopreserved at −80° C.

The cell culture supernatant extracellular vesicles purified by using nano zirconium dioxide microspheres, nano titanium dioxide microspheres and nano aluminum oxide microspheres were subjected to TEA, nanoparticle tracing analysis (NTA) and CSFE staining flow cytometry identification.

Traditional extracellular vesicle identification methods include: TEA, NTA and western blot (WB) hybridization of a specific label. Since the extracellular vesicles have tissue specificity and simple WB has no universality, we adopt a identification methods to detect the activity and quantity of the extracellular vesicles.

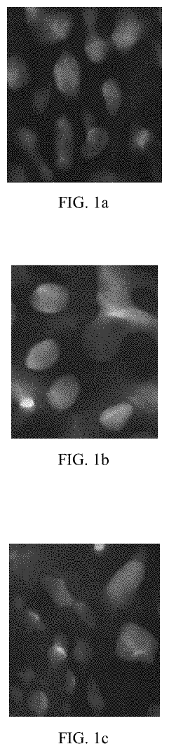

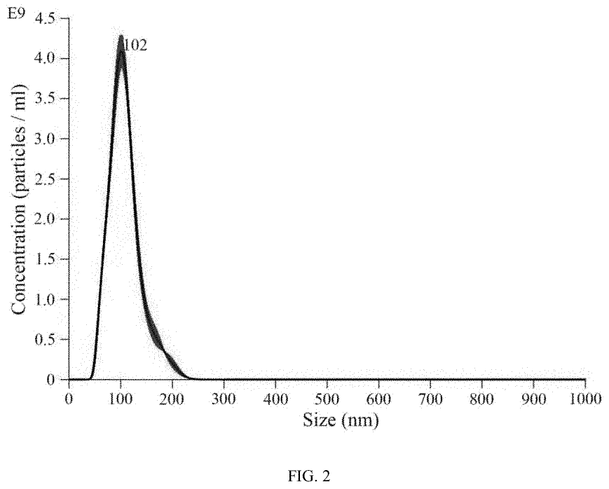

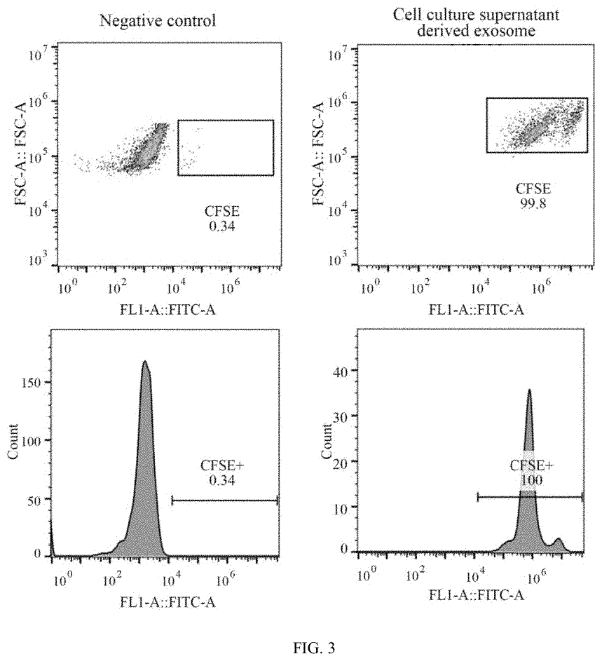

Identification results: electron micrographs are as shown in FIG. 1a, FIG. 1b and FIG. 1c. FIG. 1a is an electron micrograph of cell culture supernatant extracellular vesicles purified by using the non-magnetic nano zirconium dioxide microspheres of the present disclosure; FIG. 1b is an electron micrograph of cell culture supernatant extracellular vesicles purified by using the nano titanium dioxide microspheres of the present disclosure; FIG. 1c is an electron micrograph of cell culture supernatant extracellular vesicles purified by using the non-magnetic nano aluminum oxide microspheres of the present disclosure; NTA identification results are as shown in FIG. 2, and CSFE staining flow cytometry identification results are as shown in FIG. 3.

It can be seen from electron micrographs, NTA graphs and CSFE staining flow cytometry identification result graphs that metal oxides can reversely bind to the cell culture supernatant extracellular vesicles in the presence of a specific buffer system. Purification of extracellular vesicles with metal oxide microspheres of the present disclosure has a good purification effect. Furthermore, the metal oxide microspheres of the present disclosure can be recycled.

2. Purification of Milk-Derived Extracellular Vesicles with Non-Magnetic Nano Microspheres

Milk-derived extracellular vesicles were purified by respectively using nano zirconium dioxide microspheres, nano titanium dioxide microspheres and nano aluminum oxide microspheres obtained in examples 1-3, which specifically comprises the following steps:

Sterile pure milk within expiration date was diluted with acetic acid-sodium acetrate (200 mM, pH 4.7) in a volume ratio of 1:1, and gently and evenly mixed, and the obtained mixture was incubated for 3 h at 37° C. and centrifuged at 2500 rpm at room temperature, and supernatant was collected for standby;

1 ml of supernatant was put into a 1.5 ml EP tube, 0.5% (M/V) nano metal oxide microspheres were added based on weight, and gently and evenly mixed. The obtained mixture was placed for 15 min at room temperature and centrifuged for 5 min at 10000 g, supernatant was discarded, microspheres were resuspended with 1 ml of washing liquor (0.02% OB-2 normal saline), the obtained suspension was centrifuged for 3 min at 10000 g, supernatant was discarded, and the microspheres were repeatedly washed twice; 50 μl of elution A (100 mM sodium acetate-sodium bicarbonate, pH 10.0) was added to resuspend the microspheres, the resulting suspension was placed for 5 min at room temperature and centrifuged for 5 min at 10000 g, supernatant was transferred into a new 1.5 ml EP tube, 450 μl of elution B (normal saline) was added, the supernatant and the elution B were evenly mixed, and the obtained product was directly used for subsequent experiments or cryopreserved at −80° C.

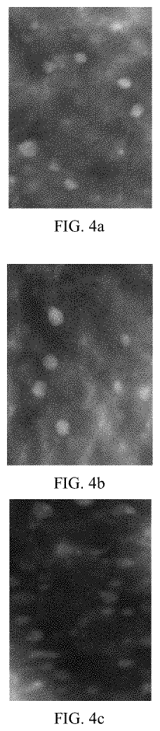

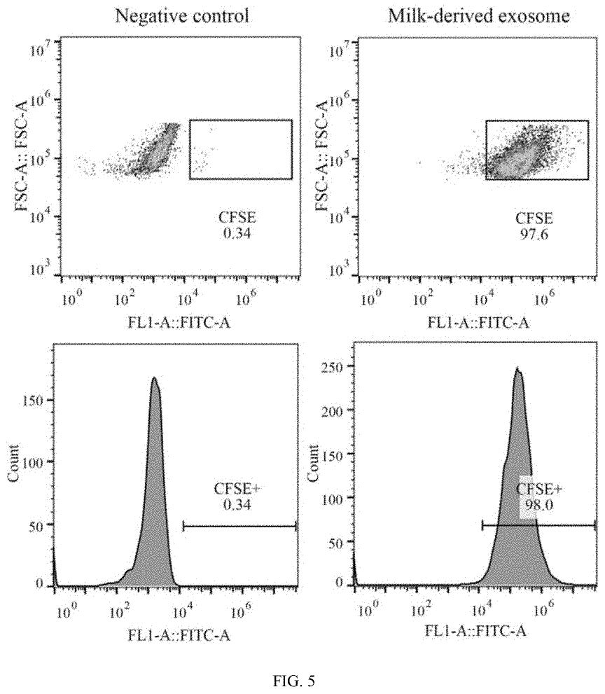

Identification results: electron micrographs are as shown in FIG. 4a, FIG. 4b and FIG. 4c. FIG. 4a is an electron micrograph of milk-derived extracellular vesicles purified by using the zirconium dioxide nano microspheres of the present disclosure; FIG. 4b is an electron micrograph of milk-derived extracellular vesicles purified by using the titanium dioxide nano microspheres of the present disclosure; FIG. 4c is an electron micrograph of milk-derived extracellular vesicles purified by using the aluminum oxide nano microspheres of the present disclosure; CSFE staining flow cytometry identification results are as shown in FIG. 5.

It can be seen from electron micrographs and CSFE staining flow cytometry identification result graphs that metal oxides can reversely bind to the milk-derived extracellular vesicles in the presence of a specific buffer system. Purification of extracellular vesicles with metal oxide microspheres of the present disclosure has a good purification effect. Furthermore, the metal oxide microspheres of the present disclosure can be recycled.

3. Purification of Human Urine Extracellular Vesicles with Non-Magnetic Nano Microspheres

human urine extracellular vesicles were purified by respectively using nano zirconium dioxide microspheres, nano titanium dioxide microspheres and nano aluminum oxide microspheres obtained in examples 1-3, which specifically comprises the following steps:

Urine was freshly collected and centrifuged for 10 min at 3000 g to remove dregs and debris. 500 μl of centrifuged urine was put into a 1.5 ml EP tube and then an equal volume of binding buffer (200 mM acetic acid-sodium acetate, pH 5.5) was added, 0.5% (M/V) nano metal oxide microspheres were added based on weight, the above materials were gently and evenly mixed, the obtained mixture was placed for 15 min at room temperature and centrifuged for 5 min at 10000 g, supernatant was discarded, the microspheres were resuspended using 1 ml of washing liquor (0.02% OB-2 normal saline), the obtained suspension was centrifuged for 3 min at 10000 g, supernatant was discarded, and the microspheres were repeatedly washed twice; 50 μl of elution A (100 mM sodium acetate-sodium bicarbonate, pH 10.0) was added to resuspend the microspheres, the resulting suspension was placed for 5 min at room temperature and centrifuged for 5 min at 10000 g, supernatant was transferred into a new 1.5 ml EP tube, 450 μl of elution B (normal saline) was added, the supernatant and the elution B were evenly mixed, and the obtained product was directly used for subsequent experiments or cryopreserved at −80° C.

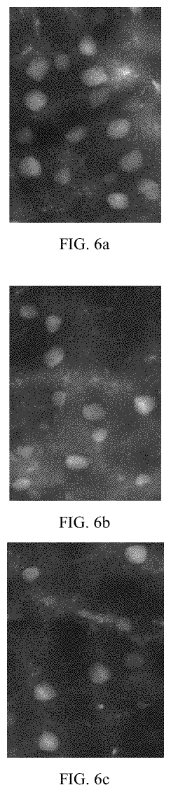

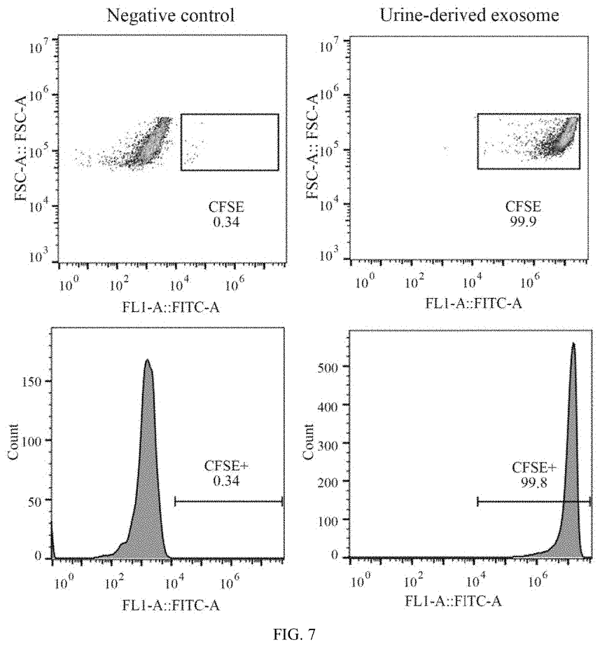

Identification results: electron micrographs are as shown in FIG. 6a, FIG. 6b and FIG. 6c. FIG. 6a is an electron micrograph of human urine-derived extracellular vesicles purified by using the zirconium dioxide nano microspheres of the present disclosure; FIG. 6b is an electron micrograph of human urine-derived extracellular vesicles purified by using the titanium dioxide nano microspheres of the present disclosure; FIG. 6c is an electron micrograph of human urine-derived extracellular vesicles purified by using the aluminum oxide nano microspheres of the present disclosure; CSFE staining flow cytometry identification results are as shown in FIG. 5.

It can be seen from electron micrographs and CSFE staining flow cytometry identification result graphs that metal oxides can reversely bind to the human urine extracellular vesicles in the presence of a specific buffer system. Purification of extracellular vesicles with metal oxide microspheres of the present disclosure has a good purification effect. Furthermore, the metal oxide microspheres of the present disclosure can be recycled.

In brief, metal oxide microspheres can reversely bind to phosphatidylserine in a biological membrane in the presence of the specific buffer system, and can purify the extracellular vesicles in a sample; the purification efficiencies of the metal oxide microspheres on extracellular vesicles in different samples are stable, the metal oxide materials can be applicable to a new method for purifying extracellular vesicles in samples.

4. Comparison on Different Magnetic Nano Microspheres to Purify Cell Culture Supernatant Extracellular Vesicles

Cell culture supernatant extracellular vesicles were purified by respectively using nano zirconium dioxide magnetic microspheres, nano titanium dioxide magnetic microspheres and nano aluminum oxide magnetic microspheres obtained in examples 4-6, which specifically comprises the following steps:

293T cell culture supernatant was freshly cultured and then centrifuged for 10 min at 3000 g to remove cell debris. 500 μl of centrifuged supernatant was placed into a 1.5 ml EP tube, an equal volume of binding buffer (200 mM acetic acid-sodium acetate, pH 5.5) was added, 0.5% (M/V) magnetic nano metal oxide microspheres were added based on weight, the above materials were gently and evenly mixed, the obtained mixture was placed for 15 min at room temperature, a sample EP tube was magnetically absorbed for 3 min on a magnetic frame, supernatant was discarded, the microspheres were resuspended using 1 ml of washing liquor (0.02% OB-2 normal saline), the sample EP tube was magnetically absorbed for 1 min on the magnetic frame, supernatant was discarded, and the microspheres were washed repeatedly twice; 50 μl of elution A (100 mM sodium acetate-sodium bicarbonate, pH 10.0) was added to resuspend the microspheres, the resulting suspension was placed for 5 min at room temperature, the sample EP tube was magnetically absorbed for 3 min on the magnetic frame, supernatant was transferred into a new 1.5 ml EP tube, 450 μl of elution B (normal saline) was added, the supernatant and the elution B were evenly mixed, the obtained product was directly used for subsequent experiments or cryopreserved at −80° C.

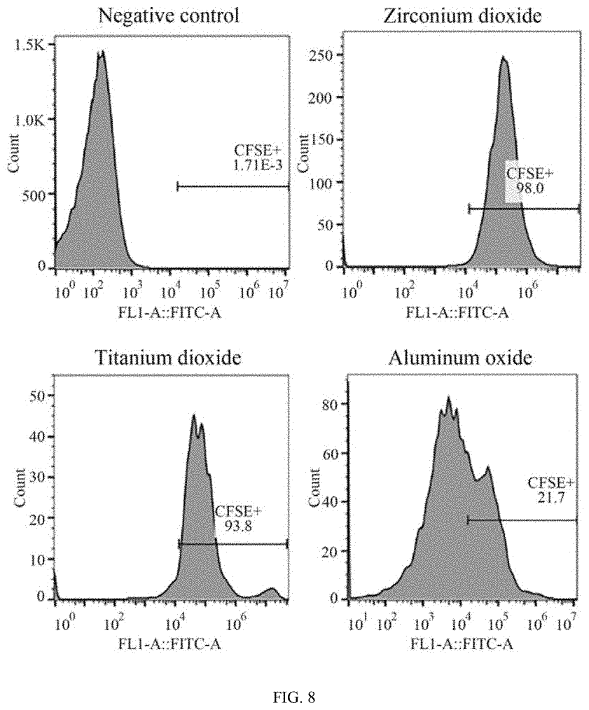

CSFE staining identification of three different metal oxide microspheres after purifying extracellular vesicles is as shown in FIG. 8.

It can be seen from FIG. 8 that different metal oxides have different binding affinities to extracellular vesicles, and possess different recovery rates of extracellular vesicles in samples under the same conditions. The purification effect of the nano zirconium dioxide magnetic microspheres is optimal (98.0%), the purification effect of the nano titanium dioxide magnetic microspheres is the second (93.8%), and the purification effect of the nano aluminum oxide magnetic microspheres is poor (23.1%).

5. Purification of Human Urine Extracellular Vesicles Via an Affine Adsorption Chromatography Column

Human urine extracellular vesicles were purified by using the porous zirconium dioxide nano microspheres obtained in example 7 as fillers to be loaded on a chromatography column, which specifically comprises the following steps:

(1) Packing

The loading amount of the required porous microspheres was calculated based on required porous microspheres loading amount (g)=external column volume of chromatography column (ml)×15%, a filler was suspended with a proper amount of absolute ethyl alcohol in a clean beaker, the external column of the chromatography column was padded with a sieve plate having a corresponding size, the sieve plate was wetted with a proper amount of absolute ethyl alcohol, the whole filler was transferred into the chromatography column at a vertical state and performed standing for 10 min, another sieve plate was padded to tightly press the filler, the chromatography column was supplemented with absolute ethyl alcohol and vertically placed on an iron stand, a container was put under the chromatography column, a cap under the chromatography column was opened to allow liquid to naturally flow out, sterile water was injected into the whole chromatography column and then naturally flew out, ½ volume of binding buffer (200 mM acetic acid-sodium acetate, pH 5.5) was added, a lower liquid outlet was sealed when the binding buffer flew through 2 column volumes, and the packing of the affinity chromatography column was completed.

(2) Purification

Collection and Pretreatment of Samples

500 ml of fresh urine on the same day was collected into a sterile container, and a sample was centrifuged for 10 min at 4° C., or passed through a filter membrane with a thickness of 0.45 μm for standby.

9 g of zirconium dioxide porous nano material was accurately weighed and loaded into a 60 ml affinity chromatography column according to step (1), and the chromatography column was balanced.

Large-Scale Purification and Identification of Extracellular Vesicles

1. 450 ml of pretreated sample was collected into a proper volume of sterile triangular flask, 50 ml of binding buffer BB (1M acetic acid-sodium acetate, pH 5.5) was added, and the above materials were gently and evenly mixed and placed for 10 min at room temperature.

2. The chromatography column was placed on the iron stand, a liquid collection vessel was placed under the chromatography column, the upper cover and the lower cap of the chromatography column were opened to allow the liquid to naturally flow out, the sample mixed solution in step 1 was added in batch to allow all the sample mixed solutions to pass through the chromatography column.

3. 60 ml of washing liquor (0.02%-OB-2 normal saline) was added into the chromatography column to allow the liquid to naturally flow out, and the chromatography column was repeatedly washed twice.

4. 20 ml of washing eluent A (100 mM sodium acetate-sodium bicarbonate, pH 10.0) was added into the chromatography column, the lower cap was put on the chromatography column when the liquid was to drop out, the chromatography column was placed for 10 min at room temperature. The lower cap was taken away from the chromatography column, and the liquid was collected into a new sterile tube.

5. 40 ml of eluent B (normal saline) was added into a collection tube and numbered after being gently and evenly mixed, and the sample extracellular vesicle in the tube was collected and used for subsequent experiments, or cryopreserved at −80° C.

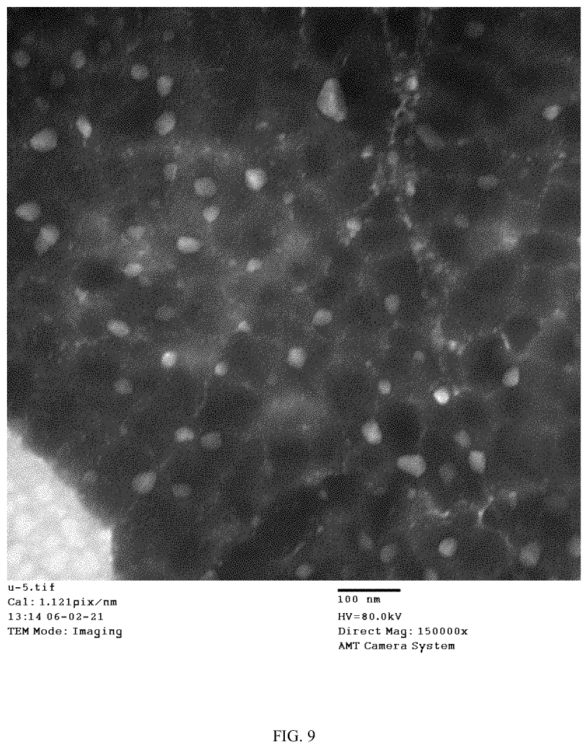

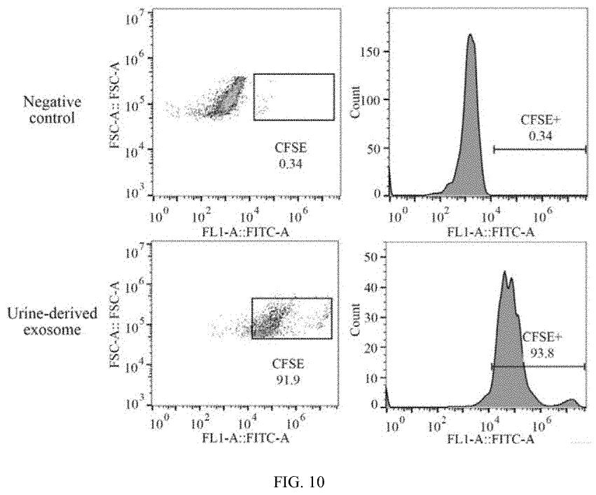

Conclusion: human urine extracellular vesicles are purified by using porous zirconium dioxide nano microspheres as the filler to be loaded onto the chromatography column. The electron microscope identification of the obtained human urine extracellular vesicles is as shown in FIG. 9, and the CSFE staining flow cytometry identification result of the obtained human urine extracellular vesicles is as shown in FIG. 10.

It can be seen from figures that the nano porous microspheres of the present disclosure can reversely bind to the human urine extracellular vesicles in the presence of the specific buffer system, large-volume samples specifically bind to extracellular vesicles through zirconium dioxide metal oxide under the action of gravity, the rest non-vesicle substances and proteins flow out without binding so as to achieve the affinity chromatography concentration of the extracellular vesicles in the sample, and the yield can be up to 90% or more.

A principle that the extracellular vesicles are purified with porous nano microspheres is as follows: an affinity chromatography capture method is based on reverse binding and elution of porous materials and phosphatidylserine (PS), and The present disclosure adopts a purification method of macroporous materials with high affinity to extracellular vesicles. Extracellular vesicles in samples are specifically adsorbed on the surfaces of porous materials under the action of binding buffer (EV-SB), while other impurities such as non-phospholipid proteins are removed without adsorption. The porous materials adsorbing the extracellular vesicles are washed with the washing liquor to remove proteins and impurities, and finally the pure extracellular vesicles are eluted with the eluent E. At present, there have no similar affinity purification products on the market so far.

The porous nano microsphere of the present disclosure has the following parameters:

Specific surface area: 120 M²/g;

Density: 0.95 g/cm³;

Particle size: 45 μm;

Recovery rate: ≥90%.

In the present disclosure, 45 μm macroporous microspheres were used to coat phosphatidylserine (PS) specific binding materials, an extracellular vesicle solid-phase purification kit was used, 9 g of affinity purification filler can at least bind to extracellular vesicles in 500 ml of sample, the purification rate can be up to 90% or more, and the eluted extracellular vesicles have high purity. Reagents such as a chelating agent are not admixed, which has a small effect on subsequent experiments.

### Comparative Example 1

Test method: the extracellular vesicles were purified with an immune adsorption method in this comparative example, and a Takara solid-phase material was labeled with potato lectin.

Test result: by comparing with results in example 2 in which nano zirconium dioxide microspheres were used to purify the extracellular vesicles, the microspheres in example 2 are more flexible, can directly and specifically bind to structural protein phosphatidylserine (PS) in a biomembrane system, and can remain the extracellular vesicles in the sample to the greatest extent. The Takara solid-phase material in comparative example 1 was labeled with potato lectin, and can only capture glycosylated extracellular vesicle subgroups. Since the lectin does not very specifically bind to glycosyl, the purified extracellular vesicles are not pure, the extracellular vesicles that are not glycosylated are lost.

### Comparative Example 2

Specific Tim4 labeled magnetic beads were used to specifically bind to phosphatidylserine (PS) through a calcium ion bridge.

Test results: by comparing with results in example 4 in which nano zirconium dioxide magnetic microspheres were used to purify the extracellular vesicles, the microspheres in example 4 adopt magnetic microspheres that have high affinity to phosphatidylserine (PS), and can directly and reversely bind to PS. Test steps are relatively simple, highly pure extracellular vesicles obtained by eluting do not contain reagents that probably affect subsequent experiments, such as the chelating agent. In comparative example 2, specific Tim4 labeled magnetic beads are used to indirectly bind to PS through the calcium ion bridge so as to cause extracellular vesicle high cost; the extracellular vesicles easily fall off after binding to magnetic beads, which leads to tedious operation steps; meanwhile, the eluted extracellular vesicles contain a large amount of chelating agents, which affects the activity of the extracellular vesicles and subsequent experiments.

The above descriptions are only specific embodiments of the present disclosure, but the protective scope of the present disclosure is not limited thereto. Those skilled in the art can readily conceive that variations or replacements within the technical scope disclosed in the present disclosure, and all of these variations or replacements should be included within the protective scope of the present disclosure. Therefore, the protective scope of the present disclosure should be based on the protective scope claimed by appended claims.

What is claimed is:

1. An extracellular vesicle purification material, wherein the purification material is a metal oxide microsphere or metal oxide magnetic bead that can reversibly bind to phosphatidylserine; the metal oxide microsphere is a non-magnetic nano microsphere or porous nano microsphere of zirconium dioxide, titanium dioxide or aluminum oxide, and the metal oxide magnetic bead is a magnetic nano microsphere of zirconium dioxide, titanium dioxide or aluminum oxide; the non-magnetic nano microsphere is a nano zirconium dioxide microsphere, a nano titanium dioxide microsphere or a nano aluminum oxide microsphere, the magnetic nano microsphere is a nano zirconium dioxide magnetic microsphere, a nano titanium dioxide magnetic microsphere or a nano aluminum oxide magnetic microsphere, and the nano porous microsphere is a porous zirconium dioxide nano microsphere, a porous titanium dioxide nano microsphere or a porous aluminum oxide nano microsphere.

2. The extracellular vesicle purification material according to claim 1, wherein a method for preparing the nano zirconium dioxide microsphere comprises the following steps:
(1) preparation of a zirconium dioxide precursor: mixing and heating zirconium dioxide powders, a first concentrated sulfuric acid and ammonium sulfate until being dissolved, adding a second concentrated sulfuric acid after the obtained mixture is placed at room temperature, and uniformly mixing to form the zirconium dioxide precursor; a weight-to-volume ratio of the zirconium dioxide powders to the first concentrated sulfuric acid and the second concentrated sulfuric acid being 0.09-0.11 g:9-11 ml:2-4 g:18-22 ml;
(2) preparation of solution A: dissolving povidone and sodium dodecylbenzenesulfonate into deionized water to be sufficiently stirred and dissolved, so as to form the solution A; a weight-to-volume ratio of povidone to sodium dodecylbenzenesulfonate to deionized water being 0.05 g:2 g:100 ml; and
(3) preparation of a nano zirconium dioxide microsphere: introducing nitrogen into the solution A obtained in step (2), adding the zirconium dioxide precursor obtained in step (1) under the state of stirring, then dropwise adding N,N-dimethylformamide, carrying out suction filtration after completion of reaction to obtain a white precipitate, washing the white precipitate with absolute ethyl alcohol, and then drying to obtain the nano zirconium dioxide microsphere; a volume ratio of the solution A to the zirconium dioxide precursor to N,N-dimethylformamide being 7:3:2.

3. The extracellular vesicle purification material according to claim 1, wherein a method for preparing the nano titanium dioxide microsphere comprises the following steps:
(1) preparation of a titanium dioxide precursor: dissolving tetrabutyl titanate into absolute ethyl alcohol, then adding deionized water, sufficiently and uniformly mixing, then adding concentrated hydrochloric acid, sealing, and then placing for 1-3 days at room temperature to obtain a light yellow transparent liquid, namely, the titanium dioxide precursor; a volume ratio of tetrabutyl titanate to absolute ethyl alcohol to deionized water to concentrated hydrochloric acid being 4:2:50:0.7;
(2) preparation of solution A: dissolving povidone and sodium dodecylbenzenesulfonate into deionized water to be sufficiently stirred and dissolved, so as to form the solution A; a weight-to-volume ratio of povidone to sodium dodecylbenzenesulfonate to deionized water being 0.05 g:2 g:100 ml; and
(3) preparation of a nano titanium dioxide microsphere: introducing nitrogen into the solution A obtained in step (2), adding the titanium dioxide precursor obtained in step (1) under the state of stirring, then dropwise adding N,N-dimethylformamide, carrying out suction filtration after completion of reaction to obtain a white precipitate, washing the white precipitate with absolute ethyl alcohol, and then drying to obtain the nano titanium dioxide microsphere; a volume ratio of the solution A to the titanium dioxide precursor to N,N-dimethylformamide being 7:3:2.

4. The extracellular vesicle purification material according to claim 1, wherein a method for preparing the nano aluminum oxide microsphere comprises the following steps:
(1) preparation of an aluminum oxide precursor: dissolving sodium metaaluminate into deionized water, and sufficiently stirring until being completely dissolved to form a uniform and transparent solution, namely, the aluminum oxide precursor; a volume ratio of sodium metaaluminate to deionized water being 0.1 g:50 ml;
(2) preparation of solution A: dissolving povidone and sodium dodecylbenzenesulfonate into deionized water to be sufficiently stirred and dissolved, so as to form the solution A; a weight-to-volume ratio of povidone to sodium dodecylbenzenesulfonate to deionized water being 0.05 g:2 g:100 ml; and
(3) preparation of a nano aluminum oxide microsphere: introducing nitrogen into the solution A obtained in step (2), adding the aluminum oxide precursor obtained in step (1) under the state of stirring, then dropwise adding N,N-dimethylformamide, carrying out suction filtration after completion of reaction to obtain a white precipitate, washing the white precipitate with absolute ethyl alcohol, and then drying to obtain the nano aluminum oxide microsphere; a volume ratio of the solution A to the aluminum oxide precursor to N,N-dimethylformamide being 7:3:2.

5. The extracellular vesicle purification material according to claim 2, wherein a method for preparing the nano zirconium dioxide magnetic microsphere comprises the following steps:
I. preparation of ferroferric oxide microsphere powders: dissolving ferric trichloride and ferrous sulfate into the solution A obtained in step (2), sufficiently stirring until being evenly mixed, dropwise adding a saturated ammonium bicarbonate solution under the state of stirring, meanwhile introducing nitrogen, stirring and heating, dropwise adding sodium hydroxide solution when the temperature is raised to 75-85° C., continuing to stir and mix under the atmosphere of nitrogen after observing that the solution turns pure black, harvesting a black precipitate via magnetic absorption, washing the black precipitate with absolute ethyl alcohol and deionized water in turn, and then drying to form the ferroferric oxide microsphere powders; the drying temperature being 60° C.; the concentration of the sodium hydroxide solution being 1M; a weight-to-volume ratio of ferric trichloride to ferrous sulfate to saturated ammonium bicarbonate solution to 1M sodium hydroxide solution being 3.24 g:2.78 g:50 ml:10 ml; and
II. preparation of a nano zirconium dioxide magnetic microsphere: adding the ferroferric oxide microsphere powders obtained in step I into the solution A obtained in step (2), introducing nitrogen, adding the zirconium dioxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, magnetically absorbing a brown precipitate, then washing the brown precipitate with absolute ethyl alcohol, and drying to obtain the nano zirconium dioxide magnetic microsphere; the drying temperature being 100° C.; the drying time being 4 h; a weight-to-volume ratio of the solution A to the ferroferric oxide microsphere powder to the zirconium dioxide precursor to N,N-dimethylformamide being 70 ml:2 g:30 ml:20 ml.

6. The extracellular vesicle purification material according to claim 3, wherein a method for preparing the nano titanium dioxide magnetic microsphere comprises the following steps:
I. preparation of ferroferric oxide microsphere powders: dissolving ferric trichloride and ferrous sulfate into the solution A obtained in step (2), sufficiently stirring until being evenly mixed, dropwise adding a saturated ammonium bicarbonate solution under the state of stirring, meanwhile introducing nitrogen, stirring and heating, dropwise adding a sodium hydroxide solution when a temperature is raised to 75-85° C., continuing to stir and mix under the atmosphere of nitrogen after observing that the solution turns pure black, harvesting a black precipitate via magnetic absorption, washing the black precipitate with absolute ethyl alcohol and deionized water in turn, and then drying to form the ferroferric oxide microsphere powders; the drying temperature being 60° C.; the concentration of the sodium hydroxide solution being 1M; a weight-to-volume ratio of ferric trichloride to ferrous sulfate to saturated ammonium bicarbonate solution to 1M sodium hydroxide solution being 3.24 g:2.78 g:50 ml:10 ml; and
II. preparation of a nano titanium dioxide magnetic microsphere: adding the ferroferric oxide microsphere powders obtained in step I into the solution A obtained in step (2), introducing nitrogen, adding the titanium dioxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, magnetically absorbing a brown precipitate, then washing the brown precipitate with absolute ethyl alcohol, and drying to obtain the nano titanium dioxide magnetic microsphere; the drying temperature being 100° C.; the drying time being 4 h; a weight-to-volume ratio of the solution A to the ferroferric oxide microsphere powder to the titanium dioxide precursor to N,N-dimethylformamide being 50 ml:2 g:50 ml:20 ml.

7. The extracellular vesicle purification material according to claim 4, wherein a method for preparing the nano aluminum oxide magnetic microsphere comprises the following steps:
I. preparation of ferroferric oxide microsphere powders: dissolving ferric trichloride and ferrous sulfate into the solution A obtained in step (2), sufficiently stirring until being evenly mixed, dropwise adding a saturated ammonium bicarbonate solution under the state of stirring, meanwhile introducing nitrogen, stirring and heating, dropwise adding a sodium hydroxide solution when a temperature is raised to 75-85° C., continuing to stir and mix under the atmosphere of nitrogen after observing that the solution turns pure black, harvesting a black precipitate via magnetic absorption, washing the black precipitate with absolute ethyl alcohol and deionized water in turn, and then drying to form the ferroferric oxide microsphere powders; the drying temperature being 60° C.; the concentration of the sodium hydroxide solution being 1M; a weight-to-volume ratio of ferric trichloride to ferrous sulfate to saturated ammonium bicarbonate solution to 1M sodium hydroxide solution being 3.24 g:2.78 g:50 ml:10 ml; and
II. preparation of a nano aluminum oxide magnetic microsphere: adding the ferroferric oxide microsphere powders obtained in step I into the solution A obtained in step (2), introducing nitrogen, adding the aluminum oxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, magnetically absorbing a brown precipitate, then washing the brown precipitate with absolute ethyl alcohol, and drying to obtain the nano aluminum oxide magnetic microsphere; the drying temperature being 100° C.; the drying time being 4 h; a weight-to-volume ratio of the solution A to the ferroferric oxide microsphere powder to the aluminum oxide precursor to N,N-dimethylformamide being 50 ml:2 g:50 ml:20 ml.

8. The extracellular vesicle purification material according to claim 2, wherein a method for preparing the porous zirconium dioxide nano microsphere comprises the following steps:
i. preparation of polystyrene microsphere powders: adding a first polystyrene and a first ammonium persulfate into the solution A obtained in step (2), sufficiently stirring and emulsifying under the atmosphere of nitrogen, dropwise adding a second polystyrene when stirring and heating to 45-55° C., adding a second ammonium persulfate after completion of dropwise addition, heating to 80-90° C., continuing to stir under the atmosphere of nitrogen until being evenly mixed, cooling to 45-55° C., then dropwise adding a sodium chloride solution, carrying out suction filtration to obtain a white precipitate, then washing the white precipitate with absolute ethyl alcohol and deionized water in turn, and then drying to form the polystyrene microsphere powders; the concentrations of ammonium persulfate being both 200 mg/ml; the concentration of the sodium chloride solution being 2M; the drying temperature being 60° C.; a weight-to-volume ratio of the first polystyrene to the first ammonium persulfate to the solution A to the second polystyrene to the second ammonium persulfate to the sodium chloride solution being 30 g:2.5 ml:200 ml:30 g:2.5 ml:20 ml; and
ii. preparation of a porous zirconium dioxide nano microsphere: adding the polystyrene microsphere powders obtained in step i into the solution A obtained in step (2), introducing nitrogen, adding the zirconium dioxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, carrying out suction filtration to obtain a white precipitate, then washing the white precipitate with absolute ethyl alcohol, and drying to form the porous zirconium dioxide nano microsphere; the drying temperature being 100° C.; the drying time being 4 h; a weight-to-volume ratio of the solution A to the polystyrene microsphere powders to the zirconium dioxide precursor to N,N-dimethylformamide being 70 ml:2 g:30 ml:20 ml.

9. The extracellular vesicle purification material according to claim 3, wherein a method for preparing the porous titanium dioxide nano microsphere comprises the following steps:
i. preparation of polystyrene microsphere powders: adding a first polystyrene and a first ammonium persulfate into the solution A obtained in step (2), sufficiently stirring and emulsifying under the atmosphere of nitrogen, dropwise adding a second polystyrene when stirring and heating to 45-55° C., adding a second ammonium persulfate after completion of dropwise addition, heating to 80-90° C., continuing to stir under the atmosphere of nitrogen until being evenly mixed, cooling to 45-55° C., then dropwise adding a sodium chloride solution, carrying out suction filtration to obtain a white precipitate, then washing the white precipitate with absolute ethyl alcohol and deionized water in turn, and then drying to form the polystyrene microsphere powders; the concentrations of ammonium persulfate being both 200 mg/ml; the concentration of the sodium chloride solution being 2M; the drying temperature being 60° C.; a weight-to-volume ratio of the first polystyrene to the first ammonium persulfate to solution A to the second polystyrene to the second ammonium persulfate to the sodium chloride solution being 30 g:2.5 ml:200 ml:30 g:2.5 ml:20 ml; and
ii. preparation of a porous titanium dioxide nano microsphere: adding the polystyrene microsphere powders obtained in step i into the solution A obtained in step (2), introducing nitrogen, adding the titanium dioxide precursor obtained in step (1) under the state of stirring, continuing to stir until being evenly mixed, dropwise adding N,N-dimethylformamide while stirring, heating to 35-45° C. after completion of dropwise addition, continuing to stir under the atmosphere of nitrogen, carrying out suction filtration to obtain a white precipitate, then washing the white precipitate with absolute ethyl alcohol, and drying to form the porous titanium dioxide nano microsphere; the drying temperature being 100° C.; the drying time being 4 h; a weight-to-volume ratio of the solution A to the polystyrene microsphere powders to the zirconium dioxide precursor to N,N-dimethylformamide being 50 ml:2 g:50 ml:20 ml.

10. An extracellular vesicle purification method, the purification method comprising: purifying extracellular vesicles using the purification material according to claim 1.
